SEQUENCE LISTING (28 sequences)

SEQ ID NO: 1 - protein, 310 aa, Mycobacterium tuberculosis
MTGPHPETESSGNRQISVAELLARQGVTGAPARRRRRRRGDSDAITVAELTGEIPIIRDDHHHAGPDAHASQSPAANGRVQVGEAAPQSPAEPVAEQVAEEPTRTVYWSQPEPRWPKSPPQDRRESGPELSEYPRPLRHTHSDRAPAGPPSGAEHMSPDPVEHYPDLWVDVLDTEVGEAEAETEVREAQPGRGERHAAAAAAGTDVEGDGAAEARVARRALDVVPTLWRGALVVLQSILAVAFGAGLFIAFDQLWRWNSIVALVLSVMVILGLVVSVRAVRKTEDIASTLIAVAVGALITLGPLALLQSG

SEQ ID NO: 2 - protein, 181 aa, Mycobacterium tuberculosis
VRVLLLGPPGAGKGTQAVKLAEKLGIPQISTGELFRRNIEEGTKLGVEAKRYLDAGDLVPSDLTNELVDDRLNNPDAANGFILDGYPRSVEQAKALHEMLERRGTDIDAVLEFRVSEEVLLERLKGRGRADDTDDVILNRMKVYRDETAPLLEYYRDQLKTVDAVGTMDEVFARALRALGK

SEQ ID NO: 3 - protein, 50 aa, Mycobacterium tuberculosis
MDFVIQWSCYLLAFLGGSAVAWVVVTLSIKRASRDEGAAEAPSAAETGAQ

SEQ ID NO: 4 - protein, 105 aa, Mycobacterium tuberculosis
VNAPLRGQVYRCDLGYGAKPWLIVSNNARNRHTADVVAVRLTTTRRTIPTWVAMGPSDPLTGYVNADNIETLGKDELGDYLGEVTPATMNKINTALATALGLPWP

SEQ ID NO: 5 - protein, 300 aa, Mycobacterium tuberculosis
MDQQSTRTDITVNVDGFWMLQALLDIRHVAPELRCRPYVSTDSNDWLNEHPGMAVMREQGIVVNDAVNEQVAARMKVLAAPDLEVVALLSRGKLLYGVIDDENQPPGSRDIPDNEFRVVLARRGQHWVSAVRVGNDITVDDVTVSDSASIAALVMDGLESIHHADPAAINAVNVPMEEMLEATKSWQESGFNVFSGGDLRRMGISAATVAALGQALSDPAAEVAVYARQYRDDAKGPSASVLSLKDGSGGRIALYQQARTAGSGEAWLAICPATPQLVQVGVKTVLDTLPYGEWKTHSRV

SEQ ID NO: 6 - protein, 228 aa, Mycobacterium tuberculosis
VRIKIFMLVTAVVLLCCSGVATAAPKTYCEELKGTDTGQACQIQMSDPAYNINISLPSYYPDQKSLENYIAQTRDKFLSAATSSTPREAPYELNITSATYQSAIPPRGTQAVVLKVYQNAGGTHPTTTYKAFDWDQAYRKPITYDTLWQADTDPLPVVFPIVQGELSKQTGQQVSIAPNAGLDPVNYQNFAVTNDGVIFFFNPGELLPEAAGPTQVLVPRSAIDSMLA

SEQ ID NO: 7 - protein, 107 aa, Mycobacterium tuberculosis
VSTYRSPDRAWQALADGTRRAIVERLAHGPLAVGELARDLPVSRPAVSQHLKVLKTARLVCDRPAGTRRVYQLDPTGLAALRTDLDRFWTRALTGYAQLIDSEGDDT

SEQ ID NO: 8 - protein, 271 aa, Mycobacterium tuberculosis
MANFTAADVKRLRELTGAGMLACKNALAETDGDFDKAVEALRIKGAKDVGKRAERATAEGLVAAKDGALIELNCETDFVAKNAEFQTLADQVVAAAAAAKPADVDALKGASIGDKTVEQAIAELSAKIGEKLELRRVAIFDGTVEAYLHRRSADLPPAVGVLVEYRGDDAAAAHAVALQIAALRARYLSRDDVPEDIVASERRIAEETARAEGKPEQALPKIVEGRLNGFFKDAVLLEQASVSDNKKTVKALLDVAGVTVTRFVRFEVGQA

SEQ ID NO: 9 - protein, 448 aa, Mycobacterium tuberculosis
LTGPEHGSASTIEILPVIGLPEFRPGDDLSAAVAAAAPWLRDGDVVVVTSKVVSKCEGRLVPAPEDPEQRDRLRRKLIEDEAVRVLARKDRTLITENRLGLVQAAAGVDGSNVGRSELALLPVDPDASAATLRAGLRERLGVTVAVVITDTMGRAWRNGQTDAAVGAAGLAVLRNYAGVRDPYGNELVVTEVAVADEIAAAADLVKGKLTATPVAVVRGFGVSDDGSTARQLLRPGANDLFWLGTAEALELGRQQAQLLRRSVRRFSTDPVPGDLVEAAVAEALTAPAPHHTRPTRFVWLQTPAIRARLLDRMKDKWRSDLTSDGLPADAIERRVARGQILYDAPEVVIPMLVPDGAHSYPDAARTDAEHTMFTVAVGAAVQALLVALAVRGLGSCWIGSTIFAADLVRDELDLPVDWEPLGAIAIGYADEPSGLRDPVPAADLLILK

SEQ ID NO: 10 - protein, 392 aa, Mycobacterium tuberculosis
MSRAFIIDPTISAIDGLYDLLGIGIPNQGGILYSSLEYFEKALEELAAAFPGDGWLGSAADKYAGKNRNHVNFFQELADLDRQLISLIHDQANAVQTTRDILEGAKKGLEFVRPVAVDLTYIPVVGHALSAAFQAPFCAGAMAVVGGALAYLVVKTLINATQLLKLLAKLAELVAAAIADIISDVADIIKGTLGEVWEFITNALNGLKELWDKLTGWVTGLFSRGWSNLESFFAGVPGLTGATSGLSQVTGLFGAAGLSASSGLAHADSLASSASLPALAGIGGGSGFGGLPSLAQVHAASTRQALRPRADGPVGAAAEQVGGQSQLVSAQGSQGMGGPVGMGGMHPSSGASKGTTTKKYSEGAAAGTEDAERAPVEADAGGGQKVLVRNVV

SEQ ID NO: 11 - protein, 106 aa, Mycobacterium tuberculosis
MRHMSETSETPTPPPHQTPKVFKAAAWVAIAAGTVFIVAVIFFTGYILGKHAGHGGFHHRQHHQHPAMMLRPGSPHGGPAAVRPGPGPGGPGQVPSSVSPPATPAP

SEQ ID NO: 12 - protein, 209 aa, Mycobacterium tuberculosis
MVRPPQTARSERTREALRQAALVRFLAQGVEATSAEQIAEDAGVSLRTFYRHFRSKHDLLFADYDAGLHWFRAALDARPADESIIDSVQAAIFSFPYDVDAVTKIASLRRGELEPSRIVRHMREVEADFADAIQAQLRRRNCDIAGAPDARLHIAVTARCVAAAVFGAMEAWMLGSDRSLGELARVCHVALESLRVGISDTWTTLTVSS

SEQ ID NO: 13 - DNA, 933 bp, Mycobacterium tuberculosis
atgacgggaccacaccccgaaacagagagctccggtaaccggcagatctcggtggccgagttgctggccaggcaaggggtcaccggcgccccggcccgacggcgccggcggcgacgcggcgatagtgacgccatcacggtcgccgagctgaccggtgagattccgatcattcgtgacgaccatcaccacgccggcccggacgcgcacgcgagccagtctccggcggctaacgggcgagtccaggttggcgaagctgccccacagtcgccggcggaaccagtcgccgagcaggttgccgaagagccaacgagaaccgtgtactggtcgcaacccgagccgcgctggcccaagtcccccccgcaggaccggcgcgagtccgggcccgagcttagcgagtacccgcggccactgcgccacacgcatagcgacagagcacccgcggggccgccgtccggtgccgaacacatgagtccggatccggtcgagcactaccccgatctctgggtggatgtcctggacaccgaggtgggcgaagcggaagccgagaccgaggtgcgcgaagcgcaacctgggcgcggcgagcgccacgccgcagcggcggcggccggcaccgacgtcgagggtgatggtgcggccgaggcgcgggttgcccgtcgtgccctggacgtggtcccgacgctgtggcgcggcgcgttggtcgtgctgcagtcgatcctggccgttgccttcggtgccgggttgttcatcgccttcgaccagttgtggcgctggaacagcatagtggcgctagtgctatcggtgatggtcatccttggcctagtggtctcggtgcgggcagtccgcaagaccgaagacatcgccagtacgttgatcgcggttgcggtgggggcgctgattaccctgggaccgctggccttgttgcaatcgggctag

SEQ ID NO: 14 - DNA, 546 bp, Mycobacterium tuberculosis
gtgagagttttgttgctgggaccgcccggggcgggcaaggggacgcaggcggtgaagctggccgagaagctcgggatcccgcagatctccaccggcgaactcttccggcgcaacatcgaagagggcaccaagctcggcgtggaagccaaacgctacttggatgccggtgacttggtgccgtccgacttgaccaatgaactcgtcgacgaccggctgaacaatccggacgcggccaacggattcatcttggatggctatccacgctcggtcgagcaggccaaggcgcttcacgagatgctcgaacgccgggggaccgacatcgacgcggtgctggagtttcgtgtgtccgaggaggtgttgttggagcgactcaaggggcgtggccgcgccgacgacaccgacgacgtcatcctcaaccggatgaaggtctaccgcgacgagaccgcgccgctgctggagtactaccgcgaccaattgaagaccgtcgacgccgtcggcaccatggacgaggtgttcgcccgtgcgttgcgggctctgggaaagtag

SEQ ID NO: 15 - DNA, 153 bp, Mycobacterium tuberculosis
atggattttgtgatccagtggtcgtgctacctgctggcgttcctggggggctcggctgttgcctgggtagtcgtcactctgtcgatcaagcgcgccagccgtgatgagggtgctgcggaggcgcccagtgcagccgagacaggcgcacagtga

SEQ ID NO: 16 - DNA, 318 bp, Mycobacterium tuberculosis
gtgaacgcgccgttgcgtggtcaggtctatcgatgcgacctcggatacggggccaaaccgtggctcatcgtctccaacaacgcccgcaaccgtcacaccgccgacgtggtggctgtgcgcctgacaacaacgcggagaaccataccgacctgggtcgccatgggccccagcgatccattgaccggatacgtcaacgcggacaacatcgagaccctcggcaaagacgagctcggtgactacctcggtgaggtcacgccggcgacgatgaacaaaatcaacacggcgctcgcgaccgcgctggggctaccgtggccatga

SEQ ID NO: 17 - DNA, 903 bp, Mycobacterium tuberculosis
atggatcaacagagtacccgcaccgacatcaccgtcaacgtcgacggcttctggatgcttcaggcgctactggatatccgccacgttgcgcctgagttacgttgccggccgtacgtctccaccgattccaatgactggctaaacgagcacccggggatggcggtcatgcgcgagcagggcattgtcgtcaacgacgcggtcaacgaacaggtcgctgcccggatgaaggtgcttgccgcacctgatcttgaagtcgtcgccctgctgtcacgcggcaagttgctgtacggggtcatagacgacgagaaccagccgccgggttcgcgtgacatccctgacaatgagttccgggtggtgttggcccggcgaggccagcactgggtgtcggcggtacgggttggcaatgacatcaccgtcgatgacgtgacggtctcggatagcgcctcgatcgccgcactggtaatggacggtctggagtcgattcaccacgccgacccagccgcgatcaacgcggtcaacgtgccaatggaggagatgctagaggcaacgaagtcgtggcaggaatcggggtttaacgtcttctccggcggagatctgcgccgaatgggcatcagtgccgcgacggtggccgcgctggggcaggcgttgtcggatcccgcggccgaggtcgcagtgtatgcgcgacagtaccgagacgacgccaagggccccagcgcctcggtgttgtcgctgaaagacggctccggtggacgcatcgcgctgtatcagcaggcgcgaacggcaggttccggcgaggcgtggctggctatctgcccggctaccccgcagttggtgcaagtaggagtgaagaccgttttggatacactgccctacggcgagtggaaaacacacagcagagtatga

SEQ ID NO: 18 - DNA, 687 bp, Mycobacterium tuberculosis
gtgcgcatcaagatcttcatgctggtcacggctgtcgttttgctctgttgttcgggtgtggccacggccgcgcccaagacctactgcgaggagttgaaaggcaccgataccggccaggcgtgccagattcaaatgtccgacccggcctacaacatcaacatcagcctgcccagttactaccccgaccagaagtcgctggaaaattacatcgcccagacgcgcgacaagttcctcagcgcggccacatcgtccactccacgcgaagccccctacgaattgaatatcacctcggccacataccagtccgcgataccgccgcgtggtacgcaggccgtggtgctcaaggtctaccagaacgccggcggcacgcacccaacgaccacgtacaaggccttcgattgggaccaggcctatcgcaagccaatcacctatgacacgctgtggcaggctgacaccgatccgctgccagtcgtcttccccattgtgcaaggtgaactgagcaagcagaccggacaacaggtatcgatagcgccgaatgccggcttggacccggtgaattatcagaacttcgcagtcacgaacgacggggtgattttcttcttcaacccgggggagttgctgcccgaagcagccggcccaacccaggtattggtcccacgttccgcgatcgactcgatgctggcctag

SEQ ID NO: 19 - DNA, 324 bp, Mycobacterium tuberculosis
gtgtccacttacagatcaccggatcgcgcttggcaggcgctggcggacggcactcgccgggccatcgtggagcggctggcgcacggcccgctggccgtcggcgagttggcccgcgacctgcccgtcagccgacccgcggtgtcacagcacctcaaagtgctcaagaccgccaggctggtgtgcgaccgccccgcgggaacacgccgcgtctaccagctcgacccgacaggccttgcggcattgcgcaccgacctcgaccggttctggacacgcgccctgactggctacgcgcagctcatcgactccgaaggagacgacacatga

SEQ ID NO: 20 - DNA, 816 bp, Mycobacterium tuberculosis
atggcgaacttcactgccgctgacgtcaagcggctgagggagctgaccggcgccggcatgctcgcctgtaagaacgcgctggccgaaaccgacggggacttcgacaaggccgtcgaggcgctgcggatcaagggcgctaaggatgtcggcaagcgtgccgagcgggctacggccgagggcttggtggcggccaaggacggcgcgctgatcgagctcaactgcgagactgactttgttgccaagaacgcggagttccaaacgctggccgaccaagtggtcgcggcggcggcagcagctaagcccgccgacgtcgacgcgctcaagggtgccagcatcggcgacaagaccgtcgagcaggcgatcgccgagctgtcggccaagatcggcgagaagctcgagctgcgtcgtgtggcgattttcgacgggaccgtggaagcctacctgcatcgacgttccgctgacctgccgccagcggtgggtgtactggtcgagtaccgcggcgacgacgcggccgccgcgcacgccgttgcgttgcaaatcgccgcgctgcgggcgcggtacctgtcccgcgacgacgtgcctgaagacatcgtggccagcgaacgccgcatcgccgaggagacggcaagggccgagggcaagccggagcaggcgctgcccaagattgtcgagggccggctgaacggcttcttcaaggatgcggtgctgcttgagcaggcgtcggtgtccgacaataagaagaccgtcaaggccctgctcgacgtggccggcgtgacggtgacacggttcgtccgcttcgaggtgggccaggcttag

SEQ ID NO: 21 - DNA, 1347 bp, Mycobacterium tuberculosis
ttgaccggccccgaacatggctccgcctcgaccatcgagatcctgcccgtcatcgggctgcccgaattccgtcccggcgacgatctgagcgccgccgtcgccgcggcggcaccgtggctacgcgacggtgacgtcgtggtggttaccagcaaggtggtgtccaaatgcgagggccggctggttccggctcccgaagaccccgagcaaagagaccgattgcgccgcaagctgatcgaggatgaggcagtgcgcgtgttggcgcgcaaggaccgcacgttgatcaccgagaatcgactcgggctggttcaggcggccgccggcgtggacggatccaacgtcggccggtccgagttagcgctgctgccggtcgatcctgacgccagtgccgcaaccttgcgcgccgggctgcgcgagcggctcggcgtcaccgtcgccgtggtcatcaccgacaccatgggacgcgcctggcgcaacggccagaccgatgccgcagtcggcgctgccggtctggcggtgctgcgcaactatgccggtgtccgcgacccatacggcaatgagttggtggtcaccgaggtcgcagtcgccgacgagatcgccgcggccgccgacttggtcaaaggcaaactgaccgcgacgccggtggcggtggtgcgtgggttcggcgtgtccgacgacggctcgacagcccggcaactgctgcggccgggcgccaacgacctgttctggctcgggaccgccgaagcgctcgagctgggtcgccagcaagcccaactgttgcgcaggtccgttcgccggtttagcaccgatccggtgccgggcgacctcgtcgaggctgcggtcgccgaggccctcaccgcgccagccccacatcacacccggccgacccgattcgtgtggctgcagacaccggccatccgcgcgcggctgctagatcggatgaaagacaagtggcggtctgatctcaccagtgacggcttgcccgccgacgcgatagaacgccgggtggcacgcggccagatcctctatgacgcacccgaagtcgtcataccgatgctggtgcccgacggagcacacagctaccccgatgccgcccgcaccgacgccgagcacaccatgttcacggtcgccgtcggagcggccgtacaagccttgctggtcgcgctggccgtgcgcgggctgggcagttgctggatcggctcgacgatctttgccgctgacctggtccgcgacgagctggacctgccagtcgactgggagccgttgggcgccatcgcgatcggatatgccgacgagccgtccgggttgcgcgacccggtgcctgccgccgatttgctgatcctgaagtga

SEQ ID NO: 22 - DNA, 1178 bp, Mycobacterium tuberculosis
tgagcagagcgttcatcatcgatccaacgatcagtgccattgacggcttgtacgaccttctggggattggaatacccaaccaagggggtatcctttactcctcactagagtacttcgaaaaagccctggaggagctggcagcagcgtttccgggtgatggctggttaggttcggccgcggacaaatacgccggcaaaaaccgcaaccacgtgaattttttccaggaactggcagacctcgatcgtcagctcatcagcctgatccacgaccaggccaacgcggtccagacgacccgcgacatcctggagggcgccaagaaaggtctcgagttcgtgcgcccggtggctgtggacctgacctacatcccggtcgtcgggcacgccctatcggccgccttccaggcgccgttttgcgcgggcgcgatggccgtagtgggcggcgcgcttgcctacttggtcgtgaaaacgctgatcaacgcgactcaactcctcaaattgcttgccaaattggcggagttggtcgcggccgccattgcggacatcatttcggatgtggcggacatcatcaagggcaccctcggagaagtgtgggagttcatcacaaacgcgctcaacggcctgaaagagctttgggacaagctcacggggtgggtgaccggactgttctctcgagggtggtcgaacctggagtccttctttgcgggcgtccccggcttgaccggcgcgaccagcggcttgtcgcaagtgactggcttgttcggtgcggccggtctgtccgcatcgtcgggcttggctcacgcggatagcctggcgagctcagccagcttgcccgccctggccggcattgggggcgggtccggttttgggggcttgccgagcctggctcaggtccatgccgcctcaactcggcaggcgctacggccccgagctgatggcccggtcggcgccgctgccgagcaggtcggcgggcagtcgcagctggtctccgcgcagggttcccaaggtatgggcggacccgtaggcatgggcggcatgcacccctcttcgggggcgtcgaaagggacgacgacgaagaagtactcggaaggcgcggcggcgggcactgaagacgccgagcgcgcgccagtcgaagctgacgcgggcggtgggcaaaaggtgctggtacgaaacgtcgtctaa

SEQ ID NO: 23 - DNA, 321 bp, Mycobacterium tuberculosis
atgcgacacatgagtgaaacatctgaaaccccaacaccaccgcctcaccagacccccaaggtgttcaaagccgcggcgtgggtagccattgccgccggaaccgtgttcatcgtggcggtcatcttcttcaccggctatatcctcggcaagcatgccggtcacggcggcttccaccaccggcaacaccaccagcacccggcgatgatgctgcgaccgggcagtccgcacggtgggcccgcggcagtgcgtcccggcccaggcccgggcgggcccggccaggtgccgtcgtccgtcagtcctcccgcgacacccgctccgtaa

SEQ ID NO: 24 - DNA, 630 bp, Mycobacterium tuberculosis
atggtccggcccccgcagacggcgcgcagcgaacgcactcgcgaggcactgcgccaggccgccctggtgcgatttctggcccagggcgtcgaggctacctcagccgagcagattgcggaggacgccggggtgtcgctgcgtactttctatcgccacttcagatccaagcacgatttgctgtttgccgactacgacgccggactgcactggtttcgcgcggcgctggatgctagaccggccgacgaatcgatcatcgattccgtgcaagcggctatcttctcgtttccttatgacgttgacgcagtgacgaagattgcgtcgttgcggcgcggcgagttggagccgagccggatcgtccgccatatgcgcgaggtggaagccgacttcgccgatgccatccaggcccaattgcggcgacgtaactgcgatatcgcgggtgcacccgatgcccgattgcacatcgccgtcaccgcacgatgcgtcgccgcggcggtgttcggcgcgatggaagcctggatgcttgggagtgaccggtcgctcggggagctggcgcgggtctgccacgtcgcactggagtcgctgcgcgtcggcatctccgacacctggaccaccctcacagtttcgtcataa

SEQ ID NO: 25 - DNA, 35 bp, Artificial Sequence
  Synthetic oligonucleotide
gaaggagatataccatgcatcatcatcatcatcat

SEQ ID NO: 26 - DNA, 20 bp, Artificial Sequence
  Synthetic oligonucleotide
tgatgatgagaacccccccc

SEQ ID NO: 27 - DNA, 169 bp, Artificial Sequence
  Synthetic oligonucleotide
cggtcacgcttgggactgccataggctggcccggtgatgccggccacgatgcgtccggcgtagaggatcgagatctcgatcccgcgaaattaatacgactcactatagggagaccacaacggtttccctctagaaataattttgtttaactttaagaaggagatatacc

SEQ ID NO: 28 - DNA, 238 bp, Artificial Sequence
  Synthetic oligonucleotide
ggggggggttctcatcatcatcatcatcattaataaaagggcgaattccagcacactggcggccgttactagtggatccggctgctaacaaagcccgaaaggaagctgagttggctgctgccaccgctgagcaataactagcataaccccttggggcctctaaacgggtcttgaggggttttttgctgaaaggaggaactatatccggagcgactcccacggcacgttggcaagctcg

(19) United States
(12) Patent Application Publication
(10) Pub. No.: US 2012/0003256 A1
(43) Pub. Date: Jan. 5, 2012

(54) TUBERCULOSIS ANTIGENS, IMMUNOGENIC COMPOSITIONS, DIAGNOSTICS AND METHODS RELATED TO THE SAME

(76) Inventors: Huiling Han, San Diego, CA (US); Dongxin K. Xu, San Diego, CA (US); Alan Greener, San Diego, CA (US)

(21) Appl. No.: 13/038,288

(22) Filed: Mar. 1, 2011

Related U.S. Application Data
(60) Provisional application No. 61/309,273, filed on Mar. 1, 2010.

(51) Int. Cl. A61K 39/04; A61P 37/04; A61P 31/06; C07H 21/04; C07K 14/35

(52) U.S. Cl. 424/190.1; 536/23.7; 530/350; 530/324

(57) ABSTRACT

Provided herein are Mtb polynucleotides, recombinant Mtb peptides and polypeptides, immunogenic Mtb antigens, and Mtb antibodies. Immunogenic compositions are also provided that may be useful as recombinant, subunit and DNA vaccines. In addition the invention provides kits comprising Mtb polynucleotides, Mtb polypeptides, Mtb antibodies, and combinations thereof.

### CROSS REFERENCE TO RELATED APPLICATIONS

The present application claims priority to U.S. Provisional Application Ser. No. 61/309,273 filed on Mar. 1, 2010 by Han et al. and entitled “TUBERCULOSIS ANTIGENS, IMMUNOGENIC COMPOSITIONS, DIAGNOSTICS AND METHODS RELATED TO THE SAME,” the entire disclosure of which is herein incorporated by reference in its entirety.

### STATEMENT REGARDING FEDERALLY SPONSORED R&D

The research leading to some of the present technology was supported, at least in part, by a grant from the National Institute of Allergy And Infectious Diseases. Accordingly, the Government may have certain rights in the technology.

### REFERENCE TO SEQUENCE LISTING

The present application is being filed along with a sequence listing in electronic format. The sequence listing is provided as a file entitled GTSYS.043A.txt, created Mar. 1, 2011 which is 34.8 KB in size. The information in the electronic format of the sequence listing is incorporated herein by reference in its entirety.

### BACKGROUND

Mycobacterium tuberculosis is the causative agent of tuberculosis, a chronic infectious disease that kills approximately 1.8 million people per year. It has been estimated that two billion people are infected with M. tuberculosis worldwide, including 10 million new cases of tuberculosis per year. In recent years there has been an unexpected rise in tuberculosis cases.

In the U.S., tuberculosis continues to be a major problem especially among the homeless, Native Americans, African-Americans, immigrants, and the elderly. Immunocompromised individuals are particularly susceptible to tuberculosis. Of the 88 million, new cases of tuberculosis projected in this decade, approximately 10% are expected to be attributable to HIV infection. The emergence of AIDS has reactivated millions of dormant cases of tuberculosis (Mtb), causing a sharp rise in the number of tuberculosis-associated deaths.

Tuberculosis is spread through the air from an infected individual to uninfected individuals through the air. For example, TB can be spread when an individual that has untreated pulmonary TB coughs, sneezes, or laughs in the vicinity of an uninfected individual. The pathogenicity and the means by which the disease is spread, illustrate the need for the development of methods to detect and diagnose the presence of Mtb.

Traditional vaccine technology suffers from the problem that it often produces various degrees of immunogenicity in different hosts. Often, the only reliably immunogenic composition is a pathogenic microorganism. Immunogenic compositions based on pathogenic microorganisms pose substantial risks, however, as the manufacture and administration of the pathogenic organism carries a risk of infection by the very pathogen the vaccine is designed to treat. Recent, well-publicized problems with influenza vaccine production illustrate the difficulties in producing large quantities of conventional vaccines and the precarious state of worldwide vaccine supplies. In light of general health concerns and the growing threat of bioterrorism, there is a need to develop recombinant and subunit vaccines capable of inducing an appropriate immune response in the context of multiple and genetically diverse hosts. This approach requires the identification of a number of specific antigenic polypeptides. One of the most difficult tasks in developing a protective or therapeutic vaccine, be it a recombinant or genetic, subunit or multi-valent vaccine, is the identification of the appropriate antigens that can stimulate the most rapid, sustained and efficacious immune responses against a particular pathogen for protection and/or therapeutic effect. This is especially challenging when the genome of the pathogen is large and screening for immunogenic antigens is tedious.

The only available vaccine for tuberculosis, BCG, is both unpredictable and highly variable in protective efficacy. Hundreds of millions of children and newborns have been vaccinated with BCG, yet this has not consistently stopped the spread of the disease. Tuberculosis has become one of the fastest spreading infectious diseases in both industrialized and developing countries worldwide. Doubtful efficacy of vaccination has spurred interest in developing effective alternatives to BCG.

The emergence of multi-drug resistant strains of M. tuberculosis, or “Mtb”, has complicated matters further, with some experts predicting a new tuberculosis epidemic. In the U.S. about 14% of M. tuberculosis isolates are resistant to at least one drug, and approximately 3% are resistant to at least two drugs. Some M. tuberculosis strains have been isolated that are resistant to as many as seven drugs in the repertoire of drugs commonly used to combat tuberculosis. Resistant strains make treatment of tuberculosis extremely difficult, leading to a mortality rate of about 90%, which is one of the reasons it has gained priority as a defined CDC—Category C Biodefense organism.

Presently, diagnostics for Mtb include sputum smears, culturing the organism, and chest X-rays. Serodiagnostic kits currently on the market exhibit poor sensitivity (less than 50%). The kits with 30-50% sensitivity exhibit low specificity; kits with higher specificity have very low sensitivity (less than 20%). Another method for diagnosing Mtb is based on the Interferon Gamma Release Assay (IGRA) and marketed as QUANTIFERON GOLD™. This method is cumbersome in that fresh blood is necessary and it is used to detect the presence of the organism, not active infection. In addition, it has poor selectivity with HVI positive and other immunocompromised patients.

Currently, there are no accurate biomarker assays for Mtb available. Tuberculosis-specific biomarkers are invaluable to help guide treatment of patients that may or may not be responding to the specific antibiotic regimen. As it often takes several weeks for clinicians to know whether the treatment course is effective; a biomarker test would enable health workers to determine the efficacy of treatment in a much shorter time frame.

In the current age, where diagnosis and treatment of tuberculosis is becoming more challenging and immunosuppressive diseases are more prevalent, new diagnostics and vaccines are essential. Thus, there is a need for developing and commercializing effective and reliable Mtb vaccines. In addition, there is a considerable need for additional diagnostic tests or tests to detect active tuberculosis in the face of other diseases such as HIV.

### SUMMARY

The embodiments disclosed herein relate to the identification and discovery of certain peptides and peptide fragments derived from M. tuberculosis that exhibit antigenic activity. Accordingly, embodiments provided herein relate to Mtb polypeptides, Mtb nucleic acids, antibodies that specifically bind to Mtb polypeptides, and compositions and kits comprising Mtb polypeptides, compositions and kits comprising Mtb nucleic acids, and compositions and kits comprising antibodies that specifically bind Mtb polypeptides.

Some embodiments provide an isolated polynucleotide encoding an antigenic Mtb polypeptide, wherein the polynucleotide is selected from the group consisting of: (a) a polynucleotide sequence comprising of any one of SEQ ID NOs: 13-24; (b) a fragment of the polynucleotide sequence of (a), wherein the fragment encodes an antigenic peptide epitope; and (c) a polynucleotide that is at least 90% identical to the full length polynucleotide of (a) or (b), wherein the polynucleotide encodes an antigenic peptide epitope. In some embodiments, the mammal is a rabbit, human, guinea pig, or mouse. In some embodiments the antigenic polypeptide is immunogenic. In some embodiments, a promoter can be operably coupled to the Mtb polynucleotide. In some embodiments, a terminator can be operably coupled to the Mtb polynucleotide.

Some embodiments provide an isolated Mtb polypeptide selected from the group consisting of: (a) a polypeptide comprising the amino acid sequence of any one of SEQ ID NOs: 1-12; (b) a fragment of the polypeptide of (a), wherein said fragment comprises an antigenic epitope; and (c) a polypeptide that is at least 90% identical to the full length polypeptide of (a) or the full length fragment of (b), wherein the polypeptide or fragment comprises an antigenic epitope. In some embodiments, an affinity tag can be operably coupled to the amino terminus or the carboxy terminus. In some embodiments, the affinity tag can be an HA tag and/or a His tag.

Some embodiments prove a composition that includes at lest one isolated Mtb polypeptide selected from the group consisting of SEQ ID NOs: 1-12, or an immunogenic fragment thereof, and a pharmaceutically acceptable carrier.

Some embodiments provide a method of generating an immune response in a mammalian host against Mtb. In some embodiments, the method includes providing to the mammalian host an immunogenic composition that includes at least one nucleic acid or fragment thereof selected from the group consisting of: (a) a polynucleotide sequence comprising of any one of SEQ ID NOs: 13-24; (b) a fragment of the polynucleotide sequence of (a), wherein the fragment encodes an antigenic peptide epitope; and (c) a polynucleotide that is at least 90% identical to the full length polynucleotide of (a) or (b), wherein the polynucleotide encodes an antigenic peptide epitope, or any combination thereof, wherein the nucleic acid encodes and expresses in vivo at least one immunogenic polypeptide. In some embodiments, the immunogenic composition includes an adjuvant.

Some embodiments provide a method of generating an immune response in a mammalian host against Mtb. The method can include the step of providing to the mammalian host an immunogenic composition that includes at least one Mtb polypeptide or immunogenic fragment thereof, selected from the group consisting of: (a) a polypeptide comprising the amino acid sequence of any one of SEQ ID NOs: 1-12; (b) a fragment of the polypeptide of (a), wherein said fragment comprises an antigenic epitope; and (c) a polypeptide that is at least 90% identical to the full length polypeptide of (a) or the full length fragment of (b), wherein the polypeptide or fragment comprises an antigenic epitope. In some embodiments, the immunogenic composition includes an adjuvant.

Some embodiments provide kits. In some embodiments, the kit can include an isolated Mtb polypeptide selected from the group consisting of: (a) a polypeptide comprising the amino acid sequence of any one of SEQ ID NOs: 1-12; (b) a fragment of the polypeptide of (a), wherein said fragment comprises an antigenic epitope; and (c) a polypeptide that is at least 90% identical to the full length polypeptide of (a) or the full length fragment of (b), wherein the polypeptide or fragment comprises an antigenic epitope, or any combination thereof; and an adjuvant. In some embodiments, the kit can include at least two of the Mtb polypeptides.

Some embodiments provide a method of detecting Mtb in a sample. In some embodiments, the method includes the steps of providing a sample, contacting the sample with at least one antibody that specifically binds an Mtb polypeptide, wherein the Mtb polypeptide selected from the group consisting of: (a) a polypeptide comprising the amino acid sequence of any one of SEQ ID NOs: 1-12; (b) a fragment of the polypeptide of (a), wherein the fragment comprises an antigenic epitope; and (c) a polypeptide that is at least 90% identical to the full length polypeptide of (a) or the full length fragment of (b), wherein the polypeptide or fragment comprises an antigenic epitope, to generate an antibody-polypeptide complex in the presence a sample comprising Mtb, and detecting the presence of the antibody-polypeptide complex. In some embodiments, the sample can be contacted with a plurality of antibodies that collectively bind to a plurality of Mtb polypeptides or immunogenic fragments thereof.

Some embodiments provide an isolated antibody that specifically binds an Mtb polypeptide selected from the group consisting of a polypeptide comprising the amino acid sequence of any one of SEQ ID NOs: 1-12. In some embodiments, the antibody is a polyclonal antibody. In some embodiments the antibody is a monoclonal antibody. In some embodiments, the antibody is a humanized antibody. In some embodiments, the antibody is a chimeric antibody. Some embodiments provide antibody fragments, wherein the antibody fragment specifically binds an Mtb polypeptide selected from the group consisting of a polypeptide comprising the amino acid sequence of any one of SEQ ID NOs: 1-12.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through use of the accompanying drawings.

FIG. 1 illustrates one method used to generate TAP Expression Fragments.

FIG. 2 displays a method of amplifying multiple genes using TAP technology, expressing said genes products, then purifying and quantifying the resulting polypeptides.

FIG. 3 demonstrates how a plurality of polypeptides from a target organism can be assayed to determine each polypeptide's ability to elicit a humoral immune response.

FIG. 4 demonstrates how a plurality of polypeptides from a target organism can be assayed to determine each polypeptide's ability to elicit a cell-mediated response.

### DETAILED DESCRIPTION

The embodiments disclosed herein relate to compositions for the detection and identification of Mycobacterium tuberculosis, as well as for the generation of an immune response against M. tuberculosis. In particular, the embodiments disclosed herein relate to, inter alia, antigenic Mycobacterium tuberculosis peptides and antigenic fragments thereof, nucleic acids encoding the antigenic peptides and antigenic fragments thereof, immunogenic compositions including the antigenic polypeptides and/or encoding nucleic acids, methods of using the polypeptides and/or fragments as biomarkers, antibodies to the polypeptides and/or fragments, as well as diagnostic kits for the detection of the peptides and nucleic acids disclosed herein.

### Mtb Polypeptides

Accordingly, some embodiments relate to polypeptides or antigenic fragments of polypeptides from M. tuberculosis (“Mtb”), including polypeptides represented in SEQ ID NO's: 1-12, or antigenic fragments thereof. Accordingly, some embodiments provide polypeptides that comprise, consist essentially of, or consist of polypeptides of SEQ ID NO: 1, SEQ ID NO: 2, SEQ ID NO: 3, SEQ ID NO: 4, SEQ ID NO:5, SEQ ID NO:6, SEQ ID NO: 7, SEQ ID NO: 8, SEQ ID NO:9, SEQ ID NO:10, SEQ ID NO:11, SEQ ID NO: 12, or variants thereof, i.e., Mtb polypeptide variants. “Mtb polypeptide variant” means an Mtb polypeptide as defined above or below having at least about 80% amino acid sequence identity with a full-length Mtb polypeptide sequence, as disclosed herein (e.g., SEQ ID NOs: 1-12, or variants thereof) or any fragment of a Mtb polypeptide sequence that is antigenic as disclosed herein. Such Mtb polypeptide variants include, for instance, Mtb polypeptides wherein one or more amino acid residues are added, or deleted, at the N- or C-terminus of the full-length Mtb amino acid sequence. Ordinarily, an Mtb polypeptide variant will have at least about 80% amino acid sequence identity, alternatively at least about 81% amino acid sequence identity, alternatively at least about 82% amino acid sequence identity, alternatively at least about 83% amino acid sequence identity, alternatively at least about 84% amino acid sequence identity, alternatively at least about 85% amino acid sequence identity, alternatively at least about 86% amino acid sequence identity, alternatively at least about 87% amino acid sequence identity, alternatively at least about 88% amino acid sequence identity, alternatively at least about 89% amino acid sequence identity, alternatively at least about 90% amino acid sequence identity, alternatively at least about 91% amino acid sequence identity, alternatively at least about 92% amino acid sequence identity, alternatively at least about 93% amino acid sequence identity, alternatively at least about 94% amino acid sequence identity, alternatively at least about 95% amino acid sequence identity, alternatively at least about 96% amino acid sequence identity, alternatively at least about 97% amino acid sequence identity, alternatively at least about 98% amino acid sequence identity and alternatively at least about 99% amino acid sequence identity to a full-length Mtb polypeptide sequence as disclosed herein (e.g., SEQ ID NO: 1-12) or any other specifically defined fragment of a full-length Mtb polypeptide sequence as disclosed herein. Ordinarily, Mtb variant polypeptides are at least about 10 amino acids in length, alternatively at least about 20 amino acids in length, alternatively at least about 30 amino acids in length, alternatively at least about 40 amino acids in length, alternatively at least about 50 amino acids in length, alternatively at least about 60 amino acids in length, alternatively at least about 70 amino acids in length, alternatively at least about 80 amino acids in length, alternatively at least about 90 amino acids in length, alternatively at least about 100 amino acids in length, alternatively at least about 150 amino acids in length, or more.

“Percent (%) amino acid sequence identity” with respect to the Mtb polypeptide sequences identified herein is defined as the percentage of amino acid residues in a candidate sequence that are identical with the amino acid residues in the specific Mtb polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, however, % amino acid sequence identity values are generated using the sequence comparison computer program ALIGN-2, wherein the complete source code for the ALIGN-2 program is available as described herein. The ALIGN-2 sequence comparison computer program was authored by Genentech, Inc. and the source code has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or may be compiled from the source code provided in Table 1 below. The ALIGN-2 program should be compiled for use on a UNIX operating system, preferably digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that programs alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. As examples of % amino acid sequence identity calculations using this method, demonstrated herein is a method to calculate the % amino acid sequence identity of the amino acid sequence designated “Comparison Protein to the amino acid sequence designated Mtb, wherein “Mtb” represents the amino acid sequence of a hypothetical Mtb polypeptide of interest, “Comparison Protein” represents the amino acid sequence of a polypeptide against which the “Mtb” polypeptide of interest is being compared, and “X, “Y” and “Z” each represent different hypothetical amino acid residues.

Unless specifically stated otherwise, all % amino acid sequence identity values used herein are obtained as described in the immediately preceding paragraph using the ALIGN-2 computer program. However, % amino acid sequence identity values may also be obtained as described below by using the WU-BLAST-2 computer program (Altschul et al., Methods in Enzymology 266:460-480 (1996)). Most of the WU-BLAST-2 search parameters are set to the default values. Those not set to default values, i.e., the adjustable parameters, are set with the following values: overlap span=1, overlap fraction=0.125, word threshold (T)=11, and scoring matrix=BLOSUM62. When WU-BLAST-2 is employed, a % amino acid sequence identity value is determined by dividing (a) the number of matching identical amino acid residues between the amino acid sequence of the Mtb polypeptide of interest having a sequence derived from the Mtb polypeptide and the comparison amino acid sequence of interest (i.e., the sequence against which the Mtb polypeptide of interest is being compared which may be a Mtb variant polypeptide) as determined by WU-BLAST-2 by (b) the total number of amino acid residues of the Mtb polypeptide of interest. For example, in the statement a polypeptide comprising an the amino acid sequence A which has or having at least 80% amino acid sequence identity to the amino acid sequence B, the amino acid sequence A is the comparison amino acid sequence of interest and the amino acid sequence B is the amino acid sequence of the Mtb polypeptide of interest.

Percent amino acid sequence identity may also be determined using the sequence comparison program NCBI-BLAST2 (Altschul et al., Nucleic Acids Res. 25:3389-3402 (1997)). The NCBI-BLAST2 sequence comparison program may be downloaded from http://www.ncbi.nlm.nih.gov or otherwise obtained from the National Institute of Health, Bethesda, Md. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program NCBI-BLAST2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

Optionally the variation is by substitution of at least one amino acid with any other amino acid in one or more of the domains of the polypeptide. Guidance in determining which amino acid residue may be inserted, substituted or deleted without adversely affecting the desired activity may be found by comparing the sequence of the polypeptide with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting variants for activity exhibited by the full-length or mature native sequence.

In particular embodiments, conservative substitutions of interest are shown below under the heading of preferred substitutions. If such substitutions result in a change in biological activity, then more substantial changes, denominated exemplary substitutions shown below, or as further described below in reference to amino acid classes, are introduced and the products screened.

| Original | Exemplary | Preferred |
|---|---|---|
| Residue | Substitutions | Substitutions |
| Ala (A) | val; leu; ile | val |
| Arg (R) | lys; gln; asn | lys |
| Asn (N) | gln; his; lys; arg | gln |
| Asp (D) | glu | glu |
| Cys (C) | ser | ser |
| Gln (Q) | asn | asn |
| Glu (E) | asp | asp |
| Gly (G) | pro; ala | ala |
| His (H) | asn; gln; lys; arg | arg |
| Ile (I) | leu; val; met; ala; phe; | leu |
| | norleucine | |
| Leu (L) | norleucine; ile; val; | ile |
| | met; ala; phe | |
| Lys (K) | arg; gln; asn | arg |
| Met (M) | leu; phe; ile | leu |
| Phe (F) | leu; val; ile; ala; tyr | leu |
| Pro (P) | ala | ala |
| Ser (S) | thr | thr |
| Thr (T) | ser | ser |
| Trp (W) | tyr; phe | tyr |
| Tyr (Y) | trp; phe; thr; ser | phe |
| Val (V) | ile; leu; met; phe; | leu |
| | ala; norleucine | |

In some embodiments, the variant polypeptides provided herein have are antigenic, comprising an epitope-bearing portion of an Mtb polypeptide described herein. Such fragments or peptides may comprise an “immunogenic epitope,” which is a part of a protein that elicits an antibody response when the entire protein is used as an immunogen. Immunogenic epitope-bearing peptides can be identified using standard methods (see, for example, Geysen et al., Proc. Nat'l Acad. Sci. USA 81:3998 (1983).

An “antigenic epitope,” which is a region of a protein molecule to which an antibody can specifically bind. Certain epitopes consist of a linear or contiguous stretch of amino acids, and the antigenicity of such an epitope is not disrupted by denaturing agents. In some embodiments, the antigenic epitope can be a conformational epitope. Conformational epitopes comprise discontinuous amino acids that come together in three dimensional conformation and interact with an antibody. It is known in the art that relatively short synthetic peptides that can mimic epitopes of a protein can be used to stimulate the production of antibodies against a protein (see, for example, Sutcliffe et al., Science 219:660 (1983)). Accordingly, antigenic epitope-bearing peptides and polypeptides of the present invention are useful to raise antibodies that bind with the polypeptides or antigenic-epitope bearing fragments described herein.

Antigenic epitope-bearing peptides and polypeptides can contain at least four to ten amino acids, at least ten to fifteen amino acids, or about 15 to about 30 amino acids of SEQ ID NOs: 2 or 5. Such epitope-bearing peptides and polypeptides can be produced by fragmenting an Mtb polypeptide, or by chemical peptide synthesis, as described herein. Moreover, epitopes can be selected by phage display of random peptide libraries (see, for example, Lane and Stephen, Curr. Opin. Immunol. 5:268 (1993), and Cortese et al., Curr. Opin. Biotechnol. 7:616 (1996)). Standard methods for identifying epitopes and producing antibodies from small peptides that comprise an epitope are described, for example, by Mole, “Epitope Mapping,” in Methods in Molecular Biology, Vol. 10, Manson (ed.), pages 105-116 (The Humana Press, Inc. 1992), Price, “Production and Characterization of Synthetic Peptide-Derived Antibodies,” in Monoclonal Antibodies: Production, Engineering, and Clinical Application, Ritter and Ladyman (eds.), pages 60-84 (Cambridge University Press 1995), and Coligan et al. (eds.), Current Protocols in Immunology, pages 9.3.1-9.3.5 and pages 9.4.1-9.4.11 (John Wiley & Sons 1997).

In some embodiments, the antigenic epitope is recognized by an antibody produced by a mammal, such as a mouse, rat, monkey, guinea pig, human, or any other mammal. Preferably, the antigenic epitope is recognized by a human antibody.

An “isolated polypeptide” is a polypeptide that is essentially free from contaminating cellular components, such as carbohydrate, lipid, or other proteinaceous impurities associated with the polypeptide in nature. Typically, a preparation of isolated polypeptide contains the polypeptide in a highly purified form, i.e., at least about 80% pure, at least about 90% pure, at least about 95% pure, greater than 95% pure, or greater than 99% pure. One way to show that a particular protein preparation contains an isolated polypeptide is by the appearance of a single band following sodium dodecyl sulfate (SDS)-polyacrylamide gel electrophoresis of the protein preparation and Coomassie Brilliant Blue staining of the gel. However, the term “isolated” does not exclude the presence of the same polypeptide in alternative physical forms, such as dimers or alternatively glycosylated or derivatized forms.

In some embodiments, the Mtb polypeptides disclosed herein can be operably coupled to an affinity tag, either at the N-terminus, the C-terminus, or at both positions. The term “affinity tag” is used herein to denote a polypeptide segment that can be attached to a second polypeptide to provide for purification or detection of the second polypeptide or provide sites for attachment of the second polypeptide to a substrate. In principal, any peptide or protein for which an antibody or other specific binding agent is available can be used as an affinity tag. Affinity tags include a poly-histidine tract, protein A (Nilsson et al., EMBO J. 4:1075 (1985); Nilsson et al., Methods Enymol. 198:3 (1991)), glutathione S transferase (Smith and Johnson, Gene 67:31 (1988)), Glu-Glu affinity tag (Grussenmeyer et al., Proc. Natl. Acad. Sci. USA 82:7952 (1985)), substance P, FLAG peptide (Hopp et al., Biotechnology 6:1204 (1988)), streptavidin binding peptide, or other antigenic epitope or binding domain. See, in general, Ford et al., Protein Expression and Purification 2:95 (1991). DNAs encoding affinity tags are available from commercial suppliers (e.g., Pharmacia Biotech, Piscataway, N.J.).

### Mtb Polynucleotides

Some embodiments relate to Mtb polynucleotides that encode Mtb polypeptides or Mtb antigenic fragments, as discussed above. Accordingly, some embodiments provide polypeptides that comprise, consist essentially of, or consist of polypeptides of SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO:21, SEQ ID NO:22, SEQ ID NO: 23, SEQ ID NO: 24, or variants thereof, i.e., Mtb polynucleotide variants.

As used herein, the terms nucleotide and polynucleotide include, for example, 3′-deoxy-2′,5′-DNA, oligodeoxyribonucleotide N3′→P5′ phosphoramidates, 2′-O-alkyl-substituted RNA, double- and single-stranded DNA, as well as double- and single-stranded RNA, DNA:RNA hybrids, and hybrids between PNAs and DNA or RNA. The terms also include known types of modifications, for example, labels which are known in the art, methylation, “caps,” substitution of one or more of the naturally occurring nucleotides with an analog, internucleotide modifications such as, for example, those with uncharged linkages (e.g., methyl phosphonates, phosphotriesters, phosphoramidates, carbamates, etc.), with negatively charged linkages (e.g., phosphorothioates, phosphorodithioates, etc.), and with positively charged linkages (e.g., aminoalklyphosphoramidates, aminoalkylphosphotriesters), those containing pendant moieties, such as, for example, proteins (including nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide or oligonucleotide.

It will be appreciated that, as used herein, the terms “nucleoside” and “nucleotide” will include those moieties which contain not only the known purine and pyrimidine bases, but also other heterocyclic bases which have been modified. Such modifications include methylated purines or pyrimidines, acylated purines or pyrimidines, or other heterocycles. Modified nucleosides or nucleotides will also include modifications on the sugar moiety, e.g., wherein one or more of the hydroxyl groups are replaced with a halogen, an aliphatic group, or are functionalized as ethers, amines, or the like. Other modifications to nucleotides or polynucleotides involve rearranging, appending, substituting for, or otherwise altering functional groups on the purine or pyrimidine base which form hydrogen bonds to a respective complementary pyrimidine or purine. The resultant modified nucleotide or polynucleotide may form a base pair with other such modified nucleotidic units but not with A, T, C, G or U. For example, guanosine (2-amino-6-oxy-9-beta.-D-ribofuranosyl-purine) may be modified to form isoguanosine (2-oxy-6-amino-9-.beta.-D-ribofuranosyl-purine). Such modification results in a nucleoside base which will no longer effectively form a standard base pair with cytosine. However, modification of cytosine (1-.beta.-D-ribofuranosyl-2-oxy-4-amino-pyrimidine) to form isocytosine (1-β-D-ribofuranosyl-2-amino-4-oxy-pyrimidine) results in a modified nucleotide which will not effectively base pair with guanosine but will form a base pair with isoguanosine. Isocytosine is available from Sigma Chemical Co. (St. Louis, Mo.); isocytidine may be prepared by the method described by Switzer et al. (1993) Biochemistry 32:10489-10496 and references cited therein; 2′-deoxy-5-methyl-isocytidine may be prepared by the method of Tor et al. (1993) J. Am. Chem. Soc. 115:4461-4467 and references cited therein; and isoguanine nucleotides may be prepared using the method described by Switzer et al. (1993), supra, and Mantsch et al. (1993) Biochem. 14:5593-5601, or by the method described U.S. Pat. No. 5,780,610 to Collins et al. The non-natural base pairs referred to as κ and π., may be synthesized by the method described in Piccirilli et al. (1990) Nature 343:33-37 for the synthesis of 2,6-diaminopyrimidine and its complement (1-methylpyrazolo[4,3]-pyrimidine-5,7-(4H,6H)-dione. Other such modified nucleotidic units which form unique base pairs have been described in Leach et al. (1992) J. Am. Chem. Soc. 114:3675-3683 and Switzer et al., supra, or will be apparent to those of ordinary skill in the art.

“Mtb variant polynucleotide” or “Mtb variant nucleic acid sequence” means a nucleic acid molecule which encodes an Mtb polypeptide as defined herein and which has at least about 80% nucleic acid sequence identity with a nucleotide acid sequence encoding a full-length Mtb polypeptide or Mtb polypeptide fragment sequence as disclosed herein. Ordinarily, an Mtb variant polynucleotide will have at least about 80% nucleic acid sequence identity, alternatively at least about 81% nucleic acid sequence identity, alternatively at least about 82% nucleic acid sequence identity, alternatively at least about 83% nucleic acid sequence identity, alternatively at least about 84% nucleic acid sequence identity, alternatively at least about 85% nucleic acid sequence identity, alternatively at least about 86% nucleic acid sequence identity, alternatively at least about 87% nucleic acid sequence identity, alternatively at least about 88% nucleic acid sequence identity, alternatively at least about 89% nucleic acid sequence identity, alternatively at least about 90% nucleic acid sequence identity, alternatively at least about 91% nucleic acid sequence identity, alternatively at least about 92% nucleic acid sequence identity, alternatively at least about 93% nucleic acid sequence identity, alternatively at least about 94% nucleic acid sequence identity, alternatively at least about 95% nucleic acid sequence identity, alternatively at least about 96% nucleic acid sequence identity, alternatively at least about 97% nucleic acid sequence identity, alternatively at least about 98% nucleic acid sequence identity and alternatively at least about 99% nucleic acid sequence identity with a nucleic acid sequence encoding a full-length Mtb polypeptide sequence as disclosed herein or any other fragment of a full-length Mtb polypeptide sequence as disclosed herein.

Ordinarily, Mtb variant polynucleotides are at least about 30 nucleotides in length, alternatively at least about 60 nucleotides in length, alternatively at least about 90 nucleotides in length, alternatively at least about 120 nucleotides in length, alternatively at least about 150 nucleotides in length, alternatively at least about 180 nucleotides in length, alternatively at least about 210 nucleotides in length, alternatively at least about 240 nucleotides in length, alternatively at least about 270 nucleotides in length, alternatively at least about 300 nucleotides in length, alternatively at least about 450 nucleotides in length, alternatively at least about 600 nucleotides in length, alternatively at least about 900 nucleotides in length, or more.

“Percent (%) nucleic acid sequence identity” with respect to Mtb-encoding nucleic acid sequences identified herein is defined as the percentage of nucleotides in a candidate sequence that are identical with the nucleotides in the Mtb nucleic acid sequence of interest, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN or Megalign (DNASTAR) software. For purposes herein, however, % nucleic acid sequence identity values are generated using the sequence comparison computer program ALIGN-2, wherein the complete source code for the ALIGN-2 program. The ALIGN-2 sequence comparison computer program was authored by Genentech, Inc. and the source code has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or may be compiled from the source code provided in Table 1 below. The ALIGN-2 program should be compiled for use on a UNIX operating system, preferably digital UNIX V4.0D. All sequence comparison parameters are set by the ALIGN-2 program and do not vary.

In situations where ALIGN-2 is employed for nucleic acid sequence comparisons, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows:

100 times the fraction W/Z

where W is the number of nucleotides scored as identical matches by the sequence alignment program ALIGN-2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C. As examples of % nucleic acid sequence identity calculations, Tables 4 and 5, demonstrate how to calculate the % nucleic acid sequence identity of the nucleic acid sequence designated “Comparison DNA” to the nucleic acid sequence designated “Mtb DNA” wherein “Mtb-DNA” represents a hypothetical Mtb-encoding nucleic acid sequence of interest, “Comparison DNA” represents the nucleotide sequence of a nucleic acid molecule against which the “Mtb-DNA” nucleic acid molecule of interest is being compared, and “N”, “L” and “V” each represent different hypothetical nucleotides.

Unless specifically stated otherwise, all % nucleic acid sequence identity values used herein are obtained as described in the immediately preceding paragraph using the ALIGN-2 computer program. However, % nucleic acid sequence identity values may also be obtained as described below by using the WU-BLAST-2 computer program (Altschul et al., Methods in Enzymology 266:460-480 (1996)). Most of the WU-BLAST-2 search parameters are set to the default values. Those not set to default values, i.e., the adjustable parameters, are set with the following values: overlap span=1, overlap fraction=0.125, word threshold (T)=11, and scoring matrix=BLOSUM62. When WU-BLAST-2 is employed, a % nucleic acid sequence identity value is determined by dividing (a) the number of matching identical nucleotides between the nucleic acid sequence of the Mtb polypeptide-encoding nucleic acid molecule of interest having a sequence derived from the native sequence Mtb polypeptide-encoding nucleic acid and the comparison nucleic acid molecule of interest (i.e., the sequence against which the Mtb polypeptide-encoding nucleic acid molecule of interest is being compared which may be a variant Mtb polynucleotide) as determined by WU-BLAST-2 by (b) the total number of nucleotides of the Mtb polypeptide-encoding nucleic acid molecule of interest. For example, in the statement “an isolated nucleic acid molecule comprising a nucleic acid sequence A which has or having at least 80% nucleic acid sequence identity to the nucleic acid sequence B, the nucleic acid sequence A is the comparison nucleic acid molecule of interest and the nucleic acid sequence B is the nucleic acid sequence of the Mtb polypeptide-encoding nucleic acid molecule of interest.

Percent nucleic acid sequence identity may also be determined using the sequence comparison program NCBI-BLAST2 (Altschul et al., Nucleic Acids Res. 25:3389-3402 (1997)). The NCBI-BLAST2 sequence comparison program may be downloaded from http://www.ncbi.nlm.nih.gov or otherwise obtained from the National Institute of Health, Bethesda, Md. NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for sequence comparisons, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows:

100 times the fraction W/Z

where W is the number of nucleotides scored as identical matches by the sequence alignment program NCBI-BLAST2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C.

Variations in the sequence of the polypeptides described herein, can be made, for example, using any of the techniques and guidelines for conservative and non-conservative mutations set forth, for instance, in U.S. Pat. No. 5,364,934. Variations may be a substitution, deletion or insertion of one or more codons encoding the polypeptide that results in a change in the amino acid sequence of the polypeptide as compared with the native sequence polypeptide.

In other embodiments, Mtb variant polynucleotides are nucleic acid molecules that encode an active Mtb polypeptide and which are capable of hybridizing, preferably under stringent hybridization and wash conditions, to nucleotide sequences encoding a full-length Mtb polypeptide as disclosed herein. Mtb variant polypeptides may be those that are encoded by an Mtb variant polynucleotide.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C.

“Moderately stringent conditions” may be identified as described by Sambrook et al., Molecular Cloning: A Laboratory Manual, New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

Variant Mtb polynucleotides are generated using any technique known to those skilled in the art, such as saturation mutagenesis, optimized directed evolution, or the like. Gene Site Saturation Mutagenesis™, or “GSSM™.” includes a method that uses degenerate oligonucleotide primers to introduce point mutations into a polynucleotide, described in detail in U.S. Pat. No. 6,673,552.

“Isolated,” when used to describe the various polypeptides disclosed herein, means polypeptide that has been identified and separated and/or recovered from a component of its natural environment. An “isolated” nucleic acid, such as an isolated Mtb polypeptide-encoding nucleic acid or other polypeptide-encoding nucleic acid is a nucleic acid molecule that is identified and separated from at least one contaminant nucleic acid molecule with which it is ordinarily associated in the natural source of the polypeptide-encoding nucleic acid. An isolated polypeptide-encoding nucleic acid molecule is other than in the form or setting in which it is found in nature. Isolated polypeptide-encoding nucleic acid molecules therefore are distinguished from the specific polypeptide-encoding nucleic acid molecule as it exists in natural cells. However, an isolated polypeptide-encoding nucleic acid molecule includes polypeptide-encoding nucleic acid molecules contained in cells that ordinarily express the polypeptide where, for example, the nucleic acid molecule is in a chromosomal location different from that of natural cells.

In some embodiments, the Mtb polynucleotides disclosed herein can be operably coupled to a regulatory element, such as a promoter, a terminator, a ribosome binding site, or the like, as discussed below.

### Vectors and Host Cells

Also provided are expression vectors and cloning vehicles comprising nucleic acids disclosed herein, e.g., sequences encoding Mtb polypeptides, e.g. SEQ ID NOs; 13-24, and variants thereof.

In some embodiments, the Mtb polynucleotides disclosed herein can be operably coupled to a promoter. As used herein, the term “promoter” refers to a DNA sequence having a regulatory function, which is recognized (directly or indirectly) and bound by a DNA-dependent RNA polymerase during the initiation of transcription. In some embodiments, the promoter can be derived from a microorganism, such as a bacterial. Exemplary bacterial promoters useful in the embodiments disclosed herein include, but are not limited to. Examples of inducible heterologous promoters used in prokaryotes include bacterial promoters such as the P_{lac}, Pₜᵣₚ, P_{tac} promoters.

Promoters are typically adjacent to the coding sequence of a gene and extend upstream from the transcription initiation site. The promoter regions may contain several short (<10 base pair) sequence elements that bind transcription factors, generally located within the first 100-200 nucleotides upstream of the transcription initiation site. Sequence elements that regulate transcription from greater distances are generally referred to as “enhancers” and may be located several hundred or thousand nucleotides away from the gene they regulate. Promoters and enhancers may be cell- and tissue-specific; they may be developmentally programmed; they may be constitutive or inducible e.g., by hormones, cytokines, antibiotics, or by physiological and metabolic states. For example, the human metallothionein (MT) promoter is upregulated by heavy metal ions and glucocorticoids. Inducible promoters and other elements may be operatively positioned to allow the inducible control or activation of expression of the desired TAP fragment. Examples of such inducible promoters and other regulatory elements include, but are not limited to, tetracycline, metallothionine, ecdysone, and other steroid-responsive promoters, rapamycin responsive promoters, and the like (see e.g., No, et al., Proc. Natl. Acad. Sci. USA, 93:3346-51 (1996); Furth, et al., Proc. Natl. Acad. Sci. USA, 91:9302-6 (1994)). Certain promoters are operative in prokaryotic cells, while different promoter sequences are required for transcription in eukaryotic cells. Additional control elements that can be used include promoters requiring specific transcription factors, such as viral promoters that may require virally encoded factors. Promoters can be selected for incorporation into TAP fragments based on the intended use of the polynucleotide, as one skilled in the art will readily appreciate. For example, if the polynucleotide encodes a polypeptide with potential utility in human cells, then a promoter capable of promoting transcription in mammalian cells can be selected. Typical mammalian promoters include muscle creatine kinase promoter, actin promoter, elongation factor promoter as well as those found in mammalian viruses such as CMV, SV40, RSV, MMV, HIV, and the like. In certain embodiments, it may be advantageous to incorporate a promoter from a plant or a plant pathogen (e.g., cauliflower mosaic virus promoter), a promoter from a fungus such as yeast (e.g., Gal 4 promoter), a promoter from a bacteria or bacterial virus, such as bacteriophage lambda, T3, T7, SP6, and the like.

The term “terminator” refers to DNA sequences, typically located at the end of a coding region, that cause RNA polymerase to terminate transcription. As used herein, the term “terminator” also encompasses terminal polynucleotide sequences that direct the processing of RNA transcripts prior to translation, such as, for example, polyadenylation signals. Any type of terminator can be used for the methods and compositions of the invention. For example, TAP terminator sequences can be derived from a prokaryote, eukaryote, or a virus, including, but not limited to animal, plant, fungal, insect, bacterial and viral sources. In one embodiment, artificial mammalian transcriptional terminator elements are used. A nonexclusive list of terminator sequences that may be used in the present invention include the SV40 transcription terminator, bovine growth hormone (BGH) terminator, synthetic terminators, rabbit .beta.-globin terminator, and the like. Terminators can also be a consecutive stretch of adenine nucleotides at the 3′ end of a TAP fragment.

Expression vectors disclosed herein may also include a selectable marker gene to allow for the selection of bacterial strains that have been transformed, e.g., genes which render the bacteria resistant to drugs such as ampicillin, chloramphenicol, erythromycin, kanamycin, neomycin and tetracycline. Selectable markers can also include biosynthetic genes, such as those in the histidine, tryptophan and leucine biosynthetic pathways. The terms “vector” and “expression cassette” as used herein can be used interchangeably and refer to a nucleotide sequence which is capable of affecting expression of a nucleic acid, e.g., a mutated nucleic acid of the invention. Expression cassettes can include at least a promoter operably linked with the polypeptide coding sequence; and, optionally, with other sequences, e.g., transcription termination signals. Additional factors necessary or helpful in effecting expression may also be used, e.g., enhancers. “Operably linked” as used herein refers to linkage of a promoter upstream from a DNA sequence such that the promoter mediates transcription of the DNA sequence. Thus, expression cassettes also include plasmids, expression vectors, recombinant viruses, any form of recombinant “naked DNA” vector, and the like. A “vector” comprises a nucleic acid which can infect, transfect, transiently or permanently transduce a cell. It will be recognized that a vector can be a naked nucleic acid, or a nucleic acid complexed with protein or lipid. The vector optionally comprises viral or bacterial nucleic acids and/or proteins, and/or membranes (e.g., a cell membrane, a viral lipid envelope, etc.). Vectors include, but are not limited to replicons (e.g., RNA replicons, bacteriophages) to which fragments of DNA may be attached and become replicated. Vectors thus include, but are not limited to RNA, autonomous self-replicating circular or linear DNA or RNA (e.g., plasmids, viruses, and the like, see, e.g., U.S. Pat. No. 5,217,879), and includes both the expression and non-expression plasmids.

The appropriate DNA sequence may be inserted into the vector by a variety of procedures. In general, the DNA sequence is ligated to the desired position in the vector following digestion of the insert and the vector with appropriate restriction endonucleases. Alternatively, blunt ends in both the insert and the vector may be ligated. A variety of cloning techniques are disclosed in Ausubel et al. Current Protocols in Molecular Biology, John Wiley 503 Sons, Inc. 1997 and Sambrook et at., Molecular Cloning: A Laboratory Manual 2nd Ed. Cold Spring Harbor Laboratory Press (1989). Such procedures and others are deemed to be within the scope of those skilled in the art. The vector may be, for example, in the form of a plasmid, a viral particle, or a phage. Other vectors include chromosomal, nonchromosomal and synthetic DNA sequences, derivatives of SV40; bacterial plasmids, phage DNA, baculovirus, yeast plasmids, vectors derived from combinations of plasmids and phage DNA, viral DNA such as vaccinia, adenovirus, fowl pox virus and pseudorabies. A variety of cloning and expression vectors for use with prokaryotic and eukaryotic hosts are described by Sambrook, et al, Molecular Cloning: A Laboratory Manual, 2nd Ed., Cold Spring Harbor, N.Y., (1989).

Also provided herein are transformed cells that comprise a nucleic acid sequence of the embodiments described herein, e.g., a sequence encoding an Mtb polypeptide or variant described herein, or an expression cassette, e.g., a vector, of the described herein. The host cell may be any of the host cells familiar to those skilled in the art, including prokaryotic cells, eukaryotic cells, such as bacterial cells, fungal cells, yeast cells, mammalian cells, insect cells, or plant cells. Exemplary bacterial cells include E. coli, Lactococcus lactis, Streptomyces, Bacillus subtilis, Bacillus cereus, Salmonella typhimurium or any species within the genera Bacillus, Streptomyces and Staphylococcus. Exemplary insect cells include Drosophila S2 and Spodoptera Sf9. Exemplary yeast cells include Pichia pastoris, Saccharomyces cerevisiae or Schizosaccharomyces pombe. Preferably, the host cell is a fungal cell. Exemplary fungal cells include species of Aspergillus, e.g. A. niger, species of Neurospora, e.g., N. crassa, and the like. Exemplary animal cells include CHO, COS or Bowes melanoma or any mouse or human cell line. The selection of an appropriate host is within the abilities of those skilled in the art. Techniques for transforming a wide variety of higher plant species are well known and described in the technical and scientific literature. See, e.g., Weising (1988) Ann. Rev. Genet. 22:421-477, U.S. Pat. No. 5,750,870. The vector can be introduced into the host cells using any of a variety of techniques, including transformation, transfection, transduction, viral infection, gene guns, or Ti-mediated gene transfer. Particular methods include calcium phosphate transfection, DEAE-Dextran mediated transfection, lipofection, or electroporation (Davis, L., Dibner, M., Battey, L, Basic Methods in Molecular Biology, (1986)). In one aspect, the nucleic acids or vectors of the invention are introduced into the cells for screening, thus, the nucleic acids enter the cells in a manner suitable for subsequent expression of the nucleic acid. The method of introduction is largely dictated by the targeted cell type. Exemplary methods include CaPO₄ precipitation, liposome fusion, lipofection (e.g., LIPOFECTIN™), electroporation, viral infection, etc. The candidate nucleic acids may stably integrate into the genome of the host cell (for example, with retroviral introduction) or may exist either transiently or stably in the cytoplasm (i.e. through the use of traditional plasmids, utilizing standard regulatory sequences, selection markers, etc.).

The constructs in host cells can be used in a conventional manner to produce the gene product encoded by the recombinant sequence. Depending upon the host employed in a recombinant production procedure, the polypeptides produced by host cells containing the vector may be glycosylated or may be non-glycosylated. Polypeptides of the invention may or may not also include an initial methionine amino acid residue. Cell-free translation systems can also be employed to produce a polypeptide of the invention. Cell-free translation systems can use mRNAs transcribed from a DNA construct comprising a promoter operably linked to a nucleic acid encoding the polypeptide or fragment thereof, hi some aspects, the DNA construct may be linearized prior to conducting an in vitro transcription reaction. The transcribed mRNA is then incubated with an appropriate cell-free translation extract, such as a rabbit reticulocyte extract, to produce the desired polypeptide or fragment thereof. The expression vectors can contain one or more selectable marker genes to provide a phenotypic trait for selection of transformed host cells such as dihydrofolate reductase or neomycin resistance for eukaryotic cell culture, or such as tetracycline or ampicillin resistance in E. coli. Host cells containing the polynucleotides of interest, e.g., nucleic acids of the invention, can be cultured in conventional nutrient media modified as appropriate for activating promoters, selecting transformants or amplifying genes. The culture conditions, such as temperature, pH and the like, are those previously used with the host cell selected for expression and will be apparent to the ordinarily skilled artisan.

Also provided are methods for overexpressing recombinant Mtb polypeptides in a cell comprising expressing a vector comprising a nucleic acid disclosed herein, e.g., a nucleic acid comprising a nucleic acid sequence with at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to SEQ ID NOs: 13-24 over a region of at least about 100 residues, or more, as described above.

Expression or overexpression of the Mtb polypeptides can be effected by any means, e.g., use of a high activity promoter, a dicistronic vector or by gene amplification of the vector. The polypeptides encoded by the nucleic acids disclosed herein can be expressed, or overexpressed, in any in vitro or in vivo expression system. Any cell culture systems can be employed to express, or over-express, recombinant protein, including bacterial, insect, yeast, fungal or mammalian cultures. Over-expression can be effected by appropriate choice of promoters, enhancers, vectors (e.g., use of replicon vectors, dicistronic vectors (see, e.g., Gurtu (1996) Biochem. Biophys. Res. Commun. 229:295-8), media, culture systems and the like. In one aspect, gene amplification using selection markers, e.g., glutamine synthetase (see, e.g., Sanders (1987) Dev. Biol. Stand. 66:55-63), in cell systems are used to overexpress the polypeptides of the invention.

In preferred embodiments, the Mtb polypeptides and Mtb polynucleotides can be expressed using TAP™ technology. With TAP™ technology, a particular polynucleotide of interest can be made transcriptionally active and ready for expression in less than one day. “TAP fragments” are transcriptionally active coding sequences prepared using TAP technology, and the two terms can be used interchangeably. TAP fragments encompass polynucleotides that can be readily expressed, for example, by transfection into animal cells or tissues by any nucleic acid transfection technique, without the need for subcloning into expression vectors or purification of plasmid DNA from bacteria. TAP fragments can be synthesized by amplification (e.g., polymerase chain reaction, or PCR) of any polynucleotide of interest using nested oligonucleotide primers. Two polynucleotide sequences are typically incorporated into TAP fragments, one of which comprises an active transcriptional promoter and the other comprises a transcriptional terminator.

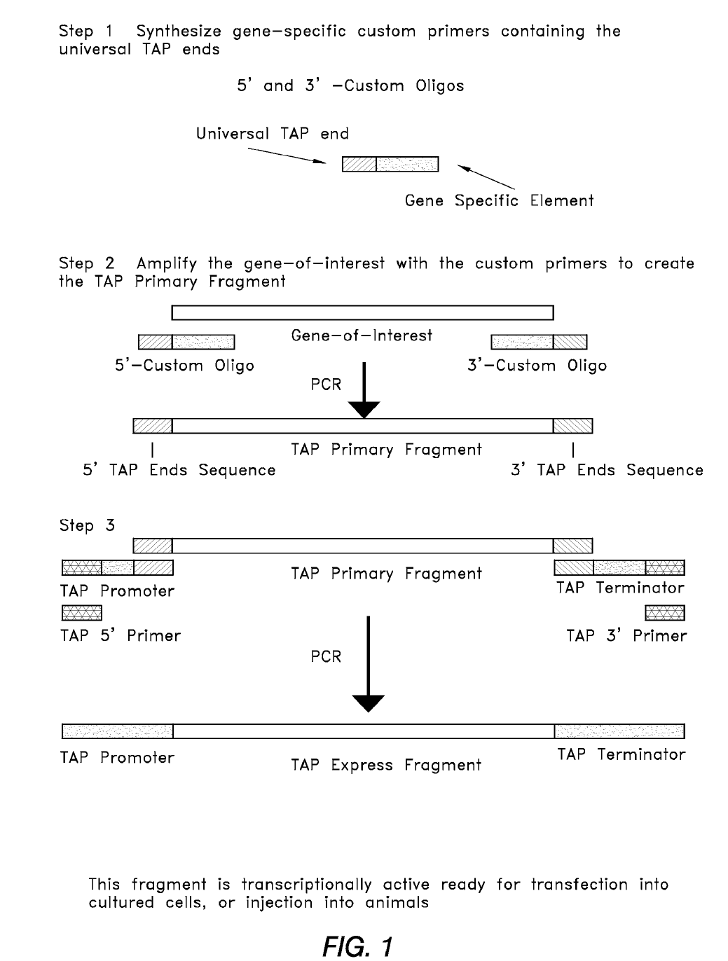

TAP fragments and methods of making the same are described in detail in U.S. Pat. No. 6,280,977, entitled “Method for Generating Transcriptionally Active DNA Fragments” which is hereby incorporated by reference in its entirety. In one embodiment, methods for creating TAP fragments include the steps of: i) designing oligonucleotide primers; ii) amplifying TAP primary fragments; and iii) amplifying TAP expression fragments. FIG. 1 illustrates one method for generating TAP fragments.

TAP fragments can be prepared using custom oligonucleotide primers designed to amplify a target polynucleotide sequence of interest from the Mtb genome. Primers complementary to the 5′ and 3′ ends of the polynucleotide of interest can be designed and synthesized using methods well known in the art, and can include any suitable number of nucleotides to permit amplification of the coding region. Typically, the polynucleotide sequence of interest is an open reading frame (ORF) that consists of an uninterrupted stretch of triplet amino acid codons, without stop codons. In certain embodiments, the polynucleotide is a Mtb polypeptide-encoding sequence.

In one embodiment, 5′-custom oligonucleotide primers of about 41, 42, 43, 44, 45 or 46 nucleotides are designed and synthesized; about 6 nucleotides of which comprise the 5′-TAP end universal sequence 5′-GAAGGAGATATACCATGCATCATCATCATCATCAT-3′ (SEQ ID NO: 25) and about 15 to 20 nucleotides are complementary to the Mtb sequence. Accordingly, the target-specific sequence can be, for example, about 15, 16, 17, 18, 19, or 20 nucleotides in length. The 5′ oligonucleotide may also incorporate a Kozak consensus sequence (A/GCCAUGG) near an ATG start codon (initiator methionine) for more efficient translation of mRNA. In one embodiment, an ATG start codon is included in the target-specific primer sequence. In another embodiment, an ATG start codon is incorporated into the custom 5′-oligonucleotide when the target sequence encoding a polypeptide of interest lacks an initiation methionine codon at its 5′ end

In one embodiment of the invention, 3′-custom oligonucleotide primers comprise about 35, 36, 37, 38, 39, 40, 41, 42, 43, 44, or 45 nucleotides; of these, about 20 nucleotides comprise the 3′-TAP end universal sequence 5′-TGATGATGAGAACCCCCCCC-3′ (SEQ ID NO: 26) and about 20 nucleotides are complementary to the target Mtb sequence. In one aspect, a stop codon sequence, can be added to the end of the target Mtb sequence to achieve proper translational termination by incorporating a TCA, TTA, or CTA into the 3′-custom oligonucleotide.

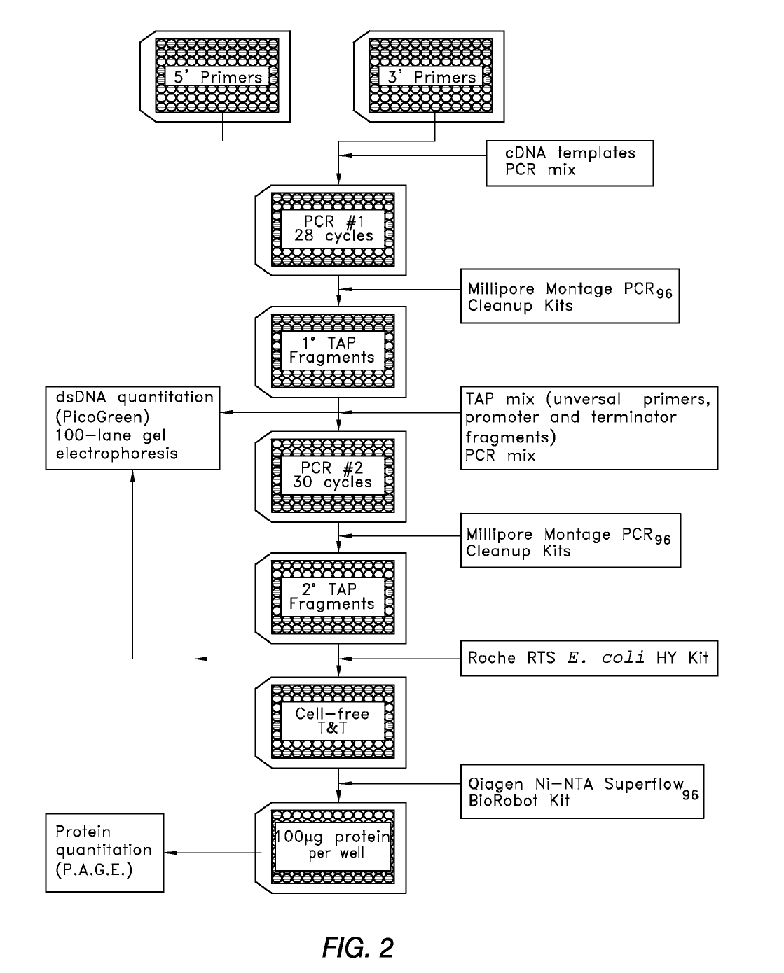

FIG. 2 illustrates a method for amplifying multiple Mtb polynucleotides using TAP technology, expressing the gene products of the resultant TAP fragments, purifying, and quantifying the resulting polypeptides. FIG. 2 further illustrates a method of preparing polypeptides, which can be assayed to identify their ability to evoke a cell-mediated or humoral immune response.

### Mtb Antibodies

Some embodiments provided herein relate to isolated Mtb antibodies, or antibody fragments, that specifically bind Mtb polypeptides. Mtb antibodies can be monoclonal or polyclonal, as discussed further below.

As used herein, the term “specifically binds” and “specific binding” refers to an antibody (or other molecule), that binds to a target such as an antigen, with greater affinity than it binds to other molecules under the specified conditions of the embodiments disclosed herein. Antibodies or antibody fragments, as known in the art, are polypeptide molecules that contain regions that can bind other molecules, such as antigens. In various embodiments of the invention, “specifically binds” may mean that an antibody or other specificity molecule, binds to a target molecule with at least about a 10⁶-fold greater affinity, preferably at least about a 10⁷-fold greater affinity, more preferably at least about a 10⁸-fold greater affinity, and most preferably at least about a 10⁹-fold greater affinity than it binds molecules unrelated to the target molecule. Typically, specific binding refers to affinities in the range of about 10⁶-fold to about 10⁹-fold greater than non-specific binding. In some embodiments, specific binding may be characterized by affinities greater than 10⁹-fold over non-specific binding. Whenever a range appears herein, as in “1-10 or one to ten, the range refers without limitation to each integer or unit of measure in the given range. Thus, by 1-10 it is meant that each of 1, 2, 3, 4, 5, 6, 7, 8, 9, 10 and any subunit in between.

Antibodies to a Mtb polypeptide can be obtained, for example, using the product of a Mtb expression vector or Mtb peptide isolated from a natural source as an antigen.

Anti-Mtb antibodies can be produced using antigenic Mtb epitope-bearing peptides and polypeptides as described herein. Antigenic epitope-bearing peptides and polypeptides of the embodiments disclosed herein contain a sequence of at least four, or between 15 to about 30 amino acids contained within SEQ ID NOs:1-12. However, peptides or polypeptides comprising a larger portion of an amino acid sequence of the embodiments disclosed herein, containing from 30 to 50 amino acids, or any length up to and including the entire amino acid sequence of a polypeptide or fragment disclosed herein, also are useful for inducing antibodies that bind with Mtb polypeptides. It is desirable that the amino acid sequence of the epitope-bearing peptide is selected to provide substantial solubility in aqueous solvents (i.e., the sequence includes relatively hydrophilic residues, while hydrophobic residues are preferably avoided). Moreover, amino acid sequences containing proline residues may be also be desirable for antibody production.

Potential antigenic sites in Mtb polypeptides, e.g., SEQ ID NOs: 1-12 can be identified using the Jameson-Wolf method, Jameson and Wolf, CABIOS 4:181, (1988), as implemented by the PROTEAN program (version 3.14) of LASERGENE (DNASTAR; Madison, Wis.). Default parameters can be used in this analysis.

The Jameson-Wolf method predicts potential antigenic determinants by combining six major subroutines for protein structural prediction. Briefly, the Hopp-Woods method, Hopp et al., Proc. Nat'l Acad. Sci. USA 78:3824 (1981), was first used to identify amino acid sequences representing areas of greatest local hydrophilicity (parameter: seven residues averaged). In the second step, Emini's method, Emini et al., J. Virology 55:836 (1985), was used to calculate surface probabilities (parameter: surface decision threshold (0.6)=1). Third, the Karplus-Schultz method, Karplus and Schultz, Naturwissenschaften 72:212 (1985), was used to predict backbone chain flexibility (parameter: flexibility threshold (0.2)=1). In the fourth and fifth steps of the analysis, secondary structure predictions were applied to the data using the methods of Chou-Fasman, Chou, “Prediction of Protein Structural Classes from Amino Acid Composition,” in Prediction of Protein Structure and the Principles of Protein Conformation, Fasman (ed.), pages 549-586 (Plenum Press 1990), and Garnier-Robson, Garnier et al., J. Mol. Biol. 120:97 (1978) (Chou-Fasman parameters: conformation table=64 proteins; α region threshold=103; β region threshold=105; Garnier-Robson parameters: α and β decision constants=0). In the sixth subroutine, flexibility parameters and hydropathy/solvent accessibility factors can be combined to determine a surface contour value, designated as the “antigenic index.” Finally, a peak broadening function can be applied to the antigenic index, which broadens major surface peaks by adding 20, 40, 60, or 80% of the respective peak value to account for additional free energy derived from the mobility of surface regions relative to interior regions. In some embodiments, this calculation is not applied any major peak that resides in a helical region, since helical regions tend to be less flexible.

Polyclonal antibodies to recombinant Mtb polypeptides or fragments disclosed herein, or to Mtb polypeptides isolated from natural sources can be prepared using methods well-known to those of skill in the art. See, for example, Green et al., “Production of Polyclonal Antisera,” in Immunochemical Protocols (Manson, ed.), pages 1-5 (Humana Press 1992), and Williams et al., “Expression of foreign proteins in E. coli using plasmid vectors and purification of specific polyclonal antibodies,” in DNA Cloning 2: Expression Systems, 2nd Edition, Glover et al. (eds.), page 15 (Oxford University Press 1995). The immunogenicity of an Mtb polypeptide can be increased through the use of an adjuvant, such as alum (aluminum hydroxide) or Freund's complete or incomplete adjuvant. Polypeptides useful for immunization also include fusion polypeptides, such as fusions of Mtb polypeptides or a portion thereof with an immunoglobulin polypeptide or with maltose binding protein. The polypeptide immunogen may be a full-length molecule or a portion thereof. If the polypeptide portion is “hapten-like,” such portion may be advantageously joined or linked to a macromolecular carrier (such as keyhole limpet hemocyanin (KLH), bovine serum albumin (BSA) or tetanus toxoid) for immunization.

Although polyclonal antibodies are typically raised in animals such as horses, cows, dogs, chicken, rats, mice, rabbits, guinea pigs, goats, or sheep, an anti-Mtb antibody of the present invention may also be derived from a subhuman primate antibody. General techniques for raising diagnostically and therapeutically useful antibodies in baboons may be found, for example, in Goldenberg et al., international patent publication No. WO 91/11465, and in Losman et al., Int. J. Cancer 46:310 (1990).

Alternatively, monoclonal anti-Mtb antibodies can be generated. Rodent monoclonal antibodies to specific antigens may be obtained by methods known to those skilled in the art (see, for example, Kohler et al., Nature 256:495 (1975), Coligan et al. (eds.), Current Protocols in Immunology, Vol. 1, pages 2.5.1-2.6.7 (John Wiley & Sons 1991) [“Coligan”], Picksley et al., “Production of monoclonal antibodies against proteins expressed in E. coli,” in DNA Cloning 2: Expression Systems, 2nd Edition, Glover et al. (eds.), page 93 (Oxford University Press 1995)).

Briefly, monoclonal antibodies can be obtained by injecting mice with a composition comprising a Mtb gene product, verifying the presence of antibody production by removing a serum sample, removing the spleen to obtain B-lymphocytes, fusing the B-lymphocytes with myeloma cells to produce hybridomas, cloning the hybridomas, selecting positive clones which produce antibodies to the antigen, culturing the clones that produce antibodies to the antigen, and isolating the antibodies from the hybridoma cultures.

In addition, an anti-Mtb antibody may be derived from a human monoclonal antibody. Human monoclonal antibodies are obtained from transgenic mice that have been engineered to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens, and the mice can be used to produce human antibody-secreting hybridomas. Methods for obtaining human antibodies from transgenic mice are described, for example, by Green et al., Nature Genet. 7:13 (1994), Lonberg et al., Nature 368:856 (1994), and Taylor et al., Int. Immun. 6:579 (1994).

Monoclonal antibodies can be isolated and purified from hybridoma cultures by a variety of well-established techniques. Such isolation techniques include affinity chromatography with Protein-A Sepharose, size-exclusion chromatography, and ion-exchange chromatography (see, for example, Coligan at pages 2.7.1-2.7.12 and pages 2.9.1-2.9.3; Baines et al., “Purification of Immunoglobulin G (IgG),” in Methods in Molecular Biology, Vol. 10, pages 79-104 (The Humana Press, Inc. 1992)).

For particular uses, it may be desirable to prepare fragments of anti-Mtb antibodies. Such antibody fragments can be obtained, for example, by proteolytic hydrolysis of the antibody. Antibody fragments can be obtained by pepsin or papain digestion of whole antibodies by conventional methods. As an illustration, antibody fragments can be produced by enzymatic cleavage of antibodies with pepsin to provide a 5S fragment denoted F(ab′)₂. This fragment can be further cleaved using a thiol reducing agent to produce 3.5S Fab′ monovalent fragments. Optionally, the cleavage reaction can be performed using a blocking group for the sulfhydryl groups that result from cleavage of disulfide linkages. As an alternative, an enzymatic cleavage using pepsin produces two monovalent Fab fragments and an Fc fragment directly. These methods are described, for example, by Goldenberg, U.S. Pat. No. 4,331,647, Nisonoff et al., Arch Biochem. Biophys. 89:230 (1960), Porter, Biochem. J. 73:119 (1959), Edelman et al., in Methods in Enzymology Vol. 1, page 422 (Academic Press 1967), and by Coligan at pages 2.8.1-2.8.10 and 2.10.-2.10.4.

Other methods of cleaving antibodies, such as separation of heavy chains to form monovalent light-heavy chain fragments, further cleavage of fragments, or other enzymatic, chemical or genetic techniques may also be used, so long as the fragments bind to the antigen that is recognized by the intact antibody.

For example, Fv fragments comprise an association of V_{H} and V_{L} chains. This association can be noncovalent, as described by Inbar et al., Proc. Nat'l Acad. Sci. USA 69:2659 (1972). Alternatively, the variable chains can be linked by an intermolecular disulfide bond or cross-linked by chemicals such as glutaraldehyde (see, for example, Sandhu, Crit. Rev. Biotech. 12:437 (1992)).

The Fv fragments may comprise V_{H} and V_{L} chains, which are connected by a peptide linker. These single-chain antigen binding proteins (scFv) are prepared by constructing a structural gene comprising DNA sequences encoding the V_{H} and V_{L} domains which are connected by an oligonucleotide. The structural gene is inserted into an expression vector, which is subsequently introduced into a host cell, such as E. coli. The recombinant host cells synthesize a single polypeptide chain with a linker peptide bridging the two V domains. Methods for producing scFvs are described, for example, by Whitlow et al., Methods: A Companion to Methods in Enzymology 2:97 (1991) (also see, Bird et al., Science 242:423 (1988), Ladner et al., U.S. Pat. No. 4,946,778, Pack et al., Bio/Technology 11:1271 (1993), and Sandhu, supra).

As an illustration, a scFV can be obtained by exposing lymphocytes to an Mtb polypeptide in vitro, and selecting antibody display libraries in phage or similar vectors (for instance, through use of immobilized or labeled Mtb protein or peptide). Genes encoding polypeptides having potential Mtb polypeptide binding domains can be obtained by screening random peptide libraries displayed on phage (phage display) or on bacteria, such as E. coli. Nucleotide sequences encoding the polypeptides can be obtained in a number of ways, such as through random mutagenesis and random polynucleotide synthesis. These random peptide display libraries can be used to screen for peptides, which interact with a known target that can be a protein or polypeptide, such as a ligand or receptor, a biological or synthetic macromolecule, or organic or inorganic substances. Techniques for creating and screening such random peptide display libraries are known in the art (Ladner et al., U.S. Pat. No. 5,223,409, Ladner et al., U.S. Pat. No. 4,946,778, Ladner et al., U.S. Pat. No. 5,403,484, Ladner et al., U.S. Pat. No. 5,571,698, and Kay et al., Phage Display of Peptides and Proteins (Academic Press, Inc. 1996)) and random peptide display libraries and kits for screening such libraries are available commercially, for instance from CLONTECH Laboratories, Inc. (Palo Alto, Calif.), Invitrogen Inc. (San Diego, Calif.), New England Biolabs, Inc. (Beverly, Mass.), and Pharmacia LKB Biotechnology Inc. (Piscataway, N.J.). Random peptide display libraries can be screened using the Mtb sequences disclosed herein to identify proteins which bind to thereto.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells (see, for example, Larrick et al., Methods: A Companion to Methods in Enzymology 2:106 (1991), Courtenay-Luck, “Genetic Manipulation of Monoclonal Antibodies,” in Monoclonal Antibodies: Production, Engineering and Clinical Application, Ritter et al. (eds.), page 166 (Cambridge University Press 1995), and Ward et al., “Genetic Manipulation and Expression of Antibodies,” in Monoclonal Antibodies: Principles and Applications, Birch et al., (eds.), page 137 (Wiley-Liss, Inc. 1995)).

Alternatively, an anti-Mtb antibody may be derived from a “humanized” monoclonal antibody. Humanized monoclonal antibodies are produced by transferring mouse complementary determining regions from heavy and light variable chains of the mouse immunoglobulin into a human variable domain. Typical residues of human antibodies are then substituted in the framework regions of the murine counterparts. The use of antibody components derived from humanized monoclonal antibodies obviates potential problems associated with the immunogenicity of murine constant regions. General techniques for cloning murine immunoglobulin variable domains are described, for example, by Orlandi et al., Proc. Nat'l Acad. Sci. USA 86:3833 (1989). Techniques for producing humanized monoclonal antibodies are described, for example, by Jones et al., Nature 321:522 (1986), Carter et al., Proc. Nat'l Acad. Sci. USA 89:4285 (1992), Sandhu, Crit. Rev. Biotech. 12:437 (1992), Singer et al., J. Immun. 150:2844 (1993), Sudhir (ed.), Antibody Engineering Protocols (Humana Press, Inc. 1995), Kelley, “Engineering Therapeutic Antibodies,” in Protein Engineering: Principles and Practice, Cleland et al. (eds.), pages 399-434 (John Wiley & Sons, Inc. 1996), and by Queen et al., U.S. Pat. No. 5,693,762 (1997).

Some embodiments provided herein relate to compositions that comprise, consist essentially of, or consist of one or more antibodies or antibody fragments that specifically bind to an Mtb polypeptide or fragment thereof disclosed herein.

For example, some embodiments relate to a composition comprising anti-Mtb antibodies disclosed herein, formulated for administration to a subject in need thereof, e.g., for passive immunization as discussed further herein. Other embodiments relate to compositions comprising anti-Mtb antibodies disclosed herein for use as a diagnostic, as disclosed further herein.

### Detection of Mtb Polynucleotides and Polypeptides

Mtb polynucleotides can be used to detect the expression of an Mtb gene in a biological sample. Such probe molecules include double-stranded nucleic acid molecules comprising the nucleotide sequence of SEQ ID NOs: 13-25, or a fragment thereof, as well as single-stranded nucleic acid molecules having the complement of the nucleotide sequence of SEQ ID NO:13-24, or a fragment thereof. Probe molecules can be DNA, RNA, oligonucleotides, and the like.

In a basic detection assay, a single-stranded probe molecule is incubated with isolated nucleic acids from a biological sample, under conditions of temperature and ionic strength that promote base pairing between the probe and target species. After separating unbound probe from hybridized molecules, the amount of hybrids is detected.

Well-established hybridization methods of DNA detection include Southern blot analysis and dot/slot blot hybridization. Nucleic acid probes can be detectably labeled with radioisotopes such as ³²P or ³⁵S. Alternatively, Mtb nucleic acids can be detected with a nonradioactive hybridization method (see, for example, Isaac (ed.), Protocols for Nucleic Acid Analysis by Nonradioactive Probes (Humana Press, Inc. 1993)). Typically, nonradioactive detection is achieved by enzymatic conversion of chromogenic or chemiluminescent substrates. Illustrative nonradioactive moieties include biotin, fluorescein, and digoxigenin.

Numerous diagnostic procedures take advantage of the polymerase chain reaction (PCR) to increase sensitivity of detection methods. Standard techniques for performing PCR are well-known (see, generally, Mathew (ed.), Protocols in Human Molecular Genetics (Humana Press, Inc. 1991), White (ed.), PCR Protocols: Current Methods and Applications (Humana Press, Inc. 1993), Cotter (ed.), Molecular Diagnosis of Cancer (Humana Press, Inc. 1996), Hanausek and Walaszek (eds.), Tumor Marker Protocols (Humana Press, Inc. 1998), Lo (ed.), Clinical Applications of PCR (Humana Press, Inc. 1998), and Meltzer (ed.), PCR in Bioanalysis (Humana Press, Inc. 1998)).

PCR amplification products can be detected using a variety of approaches. For example, PCR products can be fractionated by gel electrophoresis, and visualized by ethidium bromide staining. Alternatively, fractionated PCR products can be transferred to a membrane, hybridized with a detectably-labeled Mtb probe, and examined by autoradiography. Additional alternative approaches include the use of digoxigenin-labeled deoxyribonucleic acid triphosphates to provide chemiluminescence detection, and the C-TRAK colorimetric assay.

Detection of Mtb Proteins with Anti-Mtb Antibodies

Some embodiments relate to the use of anti-Mtb antibodies to screen biological samples in vitro for the presence of Mtb, in order to identify subjects that are infected with Mtb. In one type of in vitro assay, anti-Mtb antibodies are used in liquid phase. For example, the presence of Mtb in a biological sample can be tested by mixing the biological sample with a trace amount of labeled Mtb antibody under conditions that promote binding between Mtb and its antibody. Complexes of Mtb and anti-Mtb antibodies in the sample can be separated from the reaction mixture by contacting the complex with an immobilized protein which binds with the antibody, such as an Fc antibody or Staphylococcus protein A. The concentration of Mtb in the biological sample will be inversely proportional to the amount of labeled Mtb bound to the antibody and directly related to the amount of free-labeled Mtb. Anti-Mtb antibodies can be used in the same or a similar fashion.

Alternatively, in vitro assays can be performed in which anti-Mtb antibody is bound to a solid-phase carrier. For example, antibody can be attached to a polymer, such as aminodextran, in order to link the antibody to an insoluble support such as a polymer-coated bead, a plate or a tube. Other suitable in vitro assays will be readily apparent to those of skill in the art.

Immunochemical detection can be performed by contacting a biological sample with an anti-Mtb antibody, and then contacting the biological sample with a detectably labeled molecule that binds to the antibody. For example, the detectably labeled molecule can comprise an antibody moiety that binds to anti-Mtb antibody. Alternatively, the anti-Mtb antibody can be conjugated with avidin/streptavidin (or biotin) and the detectably labeled molecule can comprise biotin (or avidin/streptavidin). Numerous variations of this basic technique are well-known to those of skill in the art.

Alternatively, an anti-Mtb antibody can be conjugated with a detectable label to form an anti-Mtb immunoconjugate. Suitable detectable labels include, for example, a radioisotope, a fluorescent label, a chemiluminescent label, an enzyme label, a bioluminescent label or colloidal gold. Methods of making and detecting such detectably-labeled immunoconjugates are well-known to those of ordinary skill in the art, and are described in more detail below.

The detectable label can be a radioisotope that is detected by autoradiography. Isotopes that are particularly useful for the purpose of the embodiments disclosed herein are ³H, ¹²⁵I, ¹³¹I, ³⁵S and ¹⁴C.

Anti-Mtb immunoconjugates can also be labeled with a fluorescent compound. The presence of a fluorescently-labeled antibody is determined by exposing the immunoconjugate to light of the proper wavelength and detecting the resultant fluorescence. Fluorescent labeling compounds include fluorescein isothiocyanate, rhodamine, phycoerytherin, phycocyanin, allophycocyanin, o-phthaldehyde and fluorescamine.

Alternatively, anti-Mtb immunoconjugates can be detectably labeled by coupling an antibody component to a chemiluminescent compound. The presence of the chemiluminescent-tagged immunoconjugate is determined by detecting the presence of luminescence that arises during the course of a chemical reaction. Examples of chemiluminescent labeling compounds include luminol, isoluminol, an aromatic acridinium ester, an imidazole, an acridinium salt and an oxalate ester.

Similarly, a bioluminescent compound can be used to label anti-Mtb immunoconjugates disclosed herien. Bioluminescence is a type of chemiluminescence found in biological systems in which a catalytic protein increases the efficiency of the chemiluminescent reaction. The presence of a bioluminescent protein is determined by detecting the presence of luminescence. Bioluminescent compounds that are useful for labeling include luciferin, luciferase and aequorin.

Alternatively, anti-Mtb immunoconjugates can be detectably labeled by linking an anti-Mtb antibody component to an enzyme. When the anti-Mtb-enzyme conjugate is incubated in the presence of the appropriate substrate, the enzyme moiety reacts with the substrate to produce a chemical moiety, which can be detected, for example, by spectrophotometric, fluorometric or visual means. Examples of enzymes that can be used to detectably label polyspecific immunoconjugates include β-galactosidase, glucose oxidase, peroxidase and alkaline phosphatase.

Those of skill in the art will know of other suitable labels, which can be employed in accordance with the embodiments disclosed herein. The binding of marker moieties to anti-Mtb antibodies can be accomplished using standard techniques known to the art. Typical methodology in this regard is described by Kennedy et al., Clin. Chim. Acta 70:1 (1976), Schurs et al., Clin. Chim. Acta 81:1 (1977), Shih et al., Intl J. Cancer 46:1101 (1990), Stein et al., Cancer Res. 50:1330 (1990), and Coligan, supra.

Moreover, the convenience and versatility of immunochemical detection can be enhanced by using anti-Mtb antibodies that have been conjugated with avidin, streptavidin, and biotin (see, for example, Wilchek et al. (eds.), “Avidin-Biotin Technology,” Methods In Enzymology, Vol. 184 (Academic Press 1990), and Bayer et al., “Immunochemical Applications of Avidin-Biotin Technology,” in Methods In Molecular Biology, Vol. 10, Manson (ed.), pages 149-162 (The Humana Press, Inc. 1992).

Methods for performing immunoassays are well-established. See, for example, Cook and Self, “Monoclonal Antibodies in Diagnostic Immunoassays,” in Monoclonal Antibodies: Production, Engineering, and Clinical Application, Ritter and Ladyman (eds.), pages 180-208, (Cambridge University Press, 1995), Perry, “The Role of Monoclonal Antibodies in the Advancement of Immunoassay Technology,” in Monoclonal Antibodies: Principles and Applications, Birch and Lennox (eds.), pages 107-120 (Wiley-Liss, Inc. 1995), and Diamandis, Immunoassay (Academic Press, Inc. 1996).

### Kits

Some embodiments provided herein relate to kits that comprise, consist essentially of, or consist of the Mtb nucleic acids, peptides, and/or antibodies (or any combination thereof), described herein. The kits of the embodiments described herein can be useful for a variety of applications including combining reagents necessary for producing vaccine compositions, as described herein. Such vaccine compositions include the polypeptides and polynucleotides described herein as well as carriers, diluents and other pharmaceutically acceptable carriers. It should be noted, as described above, that the kits may include fragments of the nucleic acids or peptides described herein as well as combinations of the nucleic acids and/or peptides described herein. Preferably the kits include at least 2, 3, 4, 5, 6, 8, 8, 9, 10, 11, 12 or more nucleic acids or peptides described herein. Any combination of the nucleic acids or peptides can be used. In addition, the kits may include adjuvants. In addition, the kits may include instructions for preparing and administering the immunogenic compositions or vaccines.

In addition, the kits of provided herein find use as diagnostic kits. In particular, the kits find use as serodiagnostic kits. As such, the kits include at least one peptide as described herein. Preferably, however, the kits include a plurality of peptides, such as at least 2, 3, 4, 5, 6, 7, 8, 9, 10, 11, 12 or more peptides for diagnosis of Mtb infection or exposure of a subject to Mtb.

In some embodiments, the nucleic acids encoding the polypeptides find use in diagnostic kits. The nucleic acids encoding the antigenic peptides find use as probes to detect complementary nucleic acids of Mtb. However, in an alternative embodiment the kits include the polypeptides produced from the in vitro transcription-translation reaction find use in detecting antibodies from an organism, animal or patient exposed to Mtb.

The embodiments disclosed herein also provide kits for performing an immunological diagnostic assay for Mtb gene expression. Such kits comprise at least one container comprising an anti-Mtb antibody, or antibody fragment. A kit may also comprise a second container comprising one or more reagents capable of indicating the presence of Mtb antibody or antibody fragments. Examples of such indicator reagents include detectable labels such as a radioactive label, a fluorescent label, a chemiluminescent label, an enzyme label, a bioluminescent label, colloidal gold, and the like. A kit may also comprise a means for conveying to the user that Mtb antibodies or antibody fragments are used to detect Mtb polypeptide(s). For example, written instructions may state that the enclosed antibody or antibody fragment can be used to detect Mtb. The written material can be applied directly to a container, or the written material can be provided in the form of a packaging insert.

Instructions in the kits disclosed herein can be present in the kits in a variety of forms, one or more of which may be present in the kit. One form in which these instructions can be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address that may be used via the internet to access the information at a removed site. Any convenient means may be present in the kits.

### Immunogenic Compositions

Some embodiments provided herein relate to Mtb immunogenic compositions. “Immunogenic compositions” are preparations that, when administered to a human or non-human animal, elicit a humoral and/or cellular immune response. “Vaccine,” as used herein, refers to immunogenic compositions that are administered to a human or non-human patient for the prevention, amelioration or treatment of diseases, typically infectious diseases. “Traditional vaccines” or “whole vaccines” typically may be live, attenuated or killed microorganisms, such as bacteria or viruses. Vaccines also encompass preparations that elicit or stimulate an immune response that may be useful in the prevention, amelioration or treatment of non-infectious diseases. For example, a cancer cell vaccine may be administered to stimulate or supplement a patient's immune response to neoplastic disease. “Subunit vaccines” may be prepared from purified or partially purified proteins or other antigens from a microorganism, cancer cell or other vaccine target. The term “recombinant vaccine” refers to any vaccine that is prepared using recombinant DNA technology and includes certain subunit vaccines (for example, where subunits are cloned and expressed in vitro prior to administration) and “polynucleotide vaccines” such as DNA vaccines that may encode immunogenic polypeptides. Vaccines typically contain at least one immunogenic component (e.g. a cell, virus, polypeptide, polynucleotide, and the like) but may also include additional agents such as adjuvants, which may enhance or stimulate the patient's immune response to the immunogenic component. In certain embodiments, vaccines or components of vaccines may be conjugated e.g. to a polysaccharide or other molecule, to improve stability or immunogenicity of one or more vaccine components.

In some embodiments, the immunogenic compositions disclosed herein comprise one or more isolated Mtb polypeptide or antigenic-containing epitope fragments thereof, or any combination of Mtb polypeptides or fragments disclosed herein. In some embodiments, the immunogenic compositions comprise, for example, a combination of 2, 3, 4, 5, 6, 7, 8, 9, 10, 11, or 12 Mtb polypeptides (e.g., selected from the group consisting of SEQ ID NO's: 1-12) or antigenic-epitope containing fragments thereof. In some embodiments, the immunogenic composition comprises two or more Mtb polypeptides or antigenic-epitope containing fragments thereof, in the form of a single, recombinant, multivalent polypeptide, as discussed further herein.

In some embodiments, the immunogenic compositions comprise one or more isolated Mtb polynucleotides or fragments thereof, encoding an Mtb polypeptide or fragment thereof, as described herein, or a combination of Mtb polynucleotides or fragments thereof disclosed herein. In some embodiments the immunogenic compositions comprise, for example, a combination of 2, 3, 4, 5, 6, 7, 8, 9, 10, 11, or 12 Mtb polynucleotides (e.g., selected from the group consisting of SEQ ID NO's: 13-24) or antigenic-epitope encoding fragments thereof. In some embodiments, the immunogenic composition comprises two or more Mtb polynucleotides or antigenic-epitope encoding fragments thereof, operably coupled to encode a single, recombinant, multivalent polypeptide, as discussed further herein.

In some embodiments, the immunogenic compositions can include a pharmaceutically acceptable carrier. By “pharmaceutically acceptable” or “pharmacologically acceptable” is meant a material which is not biologically or otherwise undesirable, i.e., the material may be administered to an individual in a formulation or composition without causing any undesirable biological effects or interacting in a deleterious manner with any of the components of the composition in which it is contained.

The compositions described herein may contain other ingredients or compounds in addition to Mtb polypeptides and Mtb polynucleotides, including, but not limited to, various other peptides, adjuvants, binding agents, excipients such as stabilizers (to promote long term storage), emulsifiers, thickening agents, salts, preservatives, solvents, dispersion media, coatings, antibacterial and antifungal agents, isotonic and absorption delaying agents and the like. See e.g., U.S. application Ser. No. 09/929,955 and U.S. application Ser. No. 09/930,591. These compositions are suitable for treatment of animals, particularly mammals, either as a preventive measure to avoid a disease or condition or as a therapeutic to treat animals already afflicted with a disease or condition, e.g. M. tuberculosis.

Many other ingredients can also be present in the immunogenic compositions disclosed herein. For example, Mtb polypeptides and/or polynucleotides disclosed herein can be formulated in admixture with conventional excipients (e.g., pharmaceutically acceptable organic or inorganic carrier substances suitable for parenteral, enteral (e.g., oral) or topical application that do not deleteriously react with the Mtb polypeptides or polynucleotides disclosed herein). Suitable pharmaceutically acceptable carriers include, but are not limited to, water, salt solutions, alcohols, gum arabic, vegetable oils, benzyl alcohols, polyetylene glycols, gelatine, carbohydrates such as lactose, amylose or starch, magnesium stearate, talc, silicic acid, viscous paraffin, perfume oil, fatty acid monoglycerides and diglycerides, pentaerythritol fatty acid esters, hydroxy methylcellulose, polyvinyl pyrrolidone, etc. Many more suitable carriers are described in Remmington's Pharmaceutical Sciences, 15th Edition, Easton: Mack Publishing Company, pages 1405-1412 and 1461-1487 (1975) and The National Formulary XIV, 14th Edition, Washington, American Pharmaceutical Association (1975).

The Mtb polynucleotides described herein, in particular, can be formulated with or administered in conjunction with agents that increase uptake and/or expression of the polynucleotides construct by the cells relative to uptake and/or expression of the polynucleotides by the cells that occurs when the identical composition is administered in the absence of such agents. Such agents and the protocols for administering them in conjunction with gene constructs are described in PCT Patent Application Serial Number PCT/US94/00899 filed Jan. 26, 1994. Examples of such agents include: CaPO4, DEAE dextran, anionic lipids; extracellular matrix-active enzymes; saponins; lectins; estrogenic compounds and steroidal hormones; hydroxylated lower alkyls; dimethyl sulfoxide (DMSO); urea; and benzoic acid esters anilides, amidines, urethanes and the hydrochloride salts thereof, such as those of the family of local anesthetics. In addition, the gene constructs can be encapsulated within/administered in conjunction with lipids/polycationic complexes.

Vaccines can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like that do not deleteriously react with the adjuvant or the administered nucleic acid or peptide.

The effective dose and method of administration of a particular vaccine formulation can vary based on the individual patient and the type and stage of the disease, as well as other factors known to those of skill in the art. Therapeutic efficacy and toxicity of the vaccines can be determined by standard pharmaceutical procedures in cell cultures or experimental animals, e.g., ED₅₀ (the dose therapeutically effective in 50% of the population). The data obtained from cell culture assays and animal studies can be used to formulate a range of dosage for human use. The dosage of the vaccines lies preferably within a range of circulating concentrations that include the ED₅₀ with no toxicity. The dosage varies within this range depending upon the type of adjuvant derivative and HCV antigen, the dosage form employed, the sensitivity of the patient, and the route of administration.

Since many adjuvants have been on the market for several years, many dosage forms and routes of administration are known. All known dosage forms and routes of administration can be provided within the context of the embodiments described herein. Preferably, an amount of adjuvant that is effective to enhance an immune response to an antigen, e.g., an Mtb polypeptide, in an animal can be considered to be an amount that is sufficient to achieve a blood serum level of antigen approximately 0.25-12.5 μg/ml in the animal, preferably, about 2.5 μg/ml. In some embodiments, the amount of adjuvant is determined according to the body weight of the animal to be given the vaccine. Accordingly, the amount of adjuvant in a vaccine formulation can be from about 0.1-6.0 mg/kg body weight. That is, some embodiments have an amount of adjuvant that corresponds to approximately 0.1-1.0 mg/kg, 1.1-2.0 mg/kg, 2.1-3.0 mg/kg, 3.1-4.0 mg/kg, 4.1-5.0 mg/kg, and 5.1-6.0 mg/kg body weight of an animal. More conventionally, the vaccines contain approximately 0.25 mg-2000 mg of adjuvant. That is, some embodiments have approximately 250 μg, 500 μg, 1 mg, 25 mg, 50 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 1 g, 1.1 g, 1.2 g, 1.3 g, 1.4 g, 1.5 g, 1.6 g, 1.7 g, 1.8 g, 1.9 g, and 2 g of adjuvant.

As one of skill in the art will appreciate, the amount of antigens in a vaccine can vary depending on the type of antigen and its immunogenicity. The amount of antigens in the vaccine can vary accordingly. Nevertheless, as a general guide, the vaccines can have approximately 1 μg, 5 μg, 1 μg, 20 μg, 40 μg, 80 μg, 100 μg, 0.25 mg-5 mg, 5-10 mg, 10-100 mg, 100-500 mg, and upwards of 2000 mg of an antigen described herein, for example. Preferably, the amount of antigen is 0.1 μg-1 mg, desirably, 0.1 μg-100 μg, preferably 3 μg-50 μg, and, most preferably, 7 μg, 8 μg, 9 μg, 10 μg, 11 μg-20 μg, when said antigen is a nucleic acid.

In some approaches described herein, the exact amount of adjuvant and/or antigen is chosen by the individual physician in view of the patient to be treated. Further, the amounts of adjuvant can be added in combination to or separately from the same or equivalent amount of antigen and these amounts can be adjusted during a particular vaccination protocol so as to provide sufficient levels in light of patient-specific or antigen-specific considerations. In this vein, patient-specific and antigen-specific factors that can be taken into account include, but are not limited to, the severity of the disease state of the patient, age, and weight of the patient, diet, time and frequency of administration, drug combination(s), reaction sensitivities, and tolerance/response to therapy.

Vaccine compositions and immunogenic compositions comprising, consisting of, or consisting essentially of an embodied nucleic acid encoding an Mtb peptide is contemplated. These compositions typically contain an adjuvant, but do not necessarily require an adjuvant. That is, the nucleic acids and peptides described herein can function as immunogens when administered neat. The compositions described herein (e.g., the Mtb immunogens and vaccine compositions containing an adjuvant) can be manufactured in accordance with conventional methods of galenic pharmacy to produce medicinal agents for administration to animals, e.g., mammals including humans.

Various nucleic acid-based vaccines are known and it is contemplated that these compositions and approaches to immunotherapy can be augmented by reformulation with an adjuvant. By one approach, for example, a gene encoding one of the Mtb polypeptides described herein is cloned into an expression vector capable of expressing the polypeptide when introduced into a subject. The expression construct is introduced into the subject in a mixture of adjuvant or in conjunction with an adjuvant. For example, the adjuvant is administered shortly after the expression construct at the same site.

Where the antigen is to be DNA (e.g., preparation of a DNA vaccine composition), suitable promoters include Simian Virus 40 (SV40), Mouse Mammary Tumor Virus (MMTV) promoter, Human Immunodeficiency Virus (HIV) such as the HIV Long Terminal Repeat (LTR) promoter, Moloney virus, ALV, Cytomegalovirus (CMV) such as the CMV immediate early promoter, Epstein Barr Virus (EBV), Rous Sarcoma Virus (RSV) as well as promoters from human genes such as human actin, human myosin, human hemoglobin, human muscle creatine and human metalothionein. Examples of polyadenylation signals useful with some embodiments, especially in the production of a genetic vaccine for humans, include but are not limited to, SV40 polyadenylation signals and LTR polyadenylation signals. In particular, the SV40 polyadenylation signal, which is in pCEP4 plasmid (Invitrogen, San Diego Calif.), referred to as the SV40 polyadenylation signal.

More embodiments concern an immunogen comprising the an Mtb polypeptide, or a truncated, mutated, or modified version thereof, as disclosed herein. The immunogen can be provided in a substantially purified form, which means that the immunogen has been rendered substantially free of other proteins, lipids, carbohydrates or other compounds with which it naturally associates.

Some embodiments contain at least one of the nucleic acids described joined to a support. Preferably, these supports are manufactured so as to create a multimeric agent. These multimeric agents provide the Mtb polypeptide or encoding nucleic acid in such a form or in such a way that a sufficient affinity to the molecule is achieved. A multimeric agent having a Mtb polypeptide or encoding nucleic acid can be obtained by joining the desired molecule to a macromolecular support. A “support” can be a termed a carrier, a protein, a resin, a cell membrane, a capsid or portion thereof, or any macromolecular structure used to join or immobilize such molecules. Solid supports include, but are not limited to, the walls of wells of a reaction tray, test tubes, polystyrene beads, magnetic beads, nitrocellulose strips, membranes, microparticles such as latex particles, animal cells, Duracyte®, artificial cells, and others. An Mtb polypeptide or encoding nucleic acid can also be joined to inorganic carriers, such as silicon oxide material (e.g., silica gel, zeolite, diatomaceous earth or aminated glass) by, for example, a covalent linkage through a hydroxy, carboxy or amino group and a reactive group on the carrier.

In several multimeric agents, the macromolecular support has a hydrophobic surface that interacts with a portion of the Mtb polypeptide or encoding nucleic acid by a hydrophobic non-covalent interaction. In some cases, the hydrophobic surface of the support is a polymer such as plastic or any other polymer in which hydrophobic groups have been linked such as polystyrene, polyethylene or polyvinyl. Additionally, Mtb polypeptides or encoding nucleic acids can be covalently bound to carriers including proteins and oligo/polysaccharides (e.g. cellulose, starch, glycogen, chitosane or aminated sepharose). In these later multimeric agents, a reactive group on the molecule, such as a hydroxy or an amino group, is used to join to a reactive group on the carrier so as to create the covalent bond. Additional multimeric agents comprise a support that has other reactive groups that are chemically activated so as to attach Mtb polypeptides or encoding nucleic acids. For example, cyanogen bromide activated matrices, epoxy activated matrices, thio and thiopropyl gels, nitrophenyl chloroformate and N-hydroxy succinimide chlorformate linkages, or oxirane acrylic supports are used. (Sigma).

Carriers for use in the body, (i.e. for prophylactic or therapeutic applications) are desirably physiological, non-toxic and preferably, non-immunoresponsive. Suitable carriers for use in the body include poly-L-lysine, poly-D, L-alanine, liposomes, capsids that display the desired Mtb peptide or nucleic acid, and Chromosorb® (Johns-Manville Products, Denver Co.). Ligand conjugated Chromosorb® (Synsorb-Pk) has been tested in humans for the prevention of hemolytic-uremic syndrome and was reported as not presenting adverse reactions. (Armstrong et al. J. Infectious Diseases 171:1042-1045 (1995)). For some embodiments, a “naked” carrier (i.e., lacking an attached Mtb polypeptide or encoding nucleic acid) that has the capacity to attach an Mtb polypeptide or encoding nucleic acid in the body of a organism is administered. By this approach, a “prodrug-type” therapy is envisioned in which the naked carrier is administered separately from the Mtb polypeptide or encoding nucleic acid and, once both are in the body of the organism, the carrier and NS3/4A chimeric polypeptide or encoding nucleic acid are assembled into a multimeric complex.

The insertion of linkers of an appropriate length between the Mtb or encoding nucleic acid and the support are also contemplated so as to encourage greater flexibility of the Mtb polypeptide, encoding nucleic acid, hybrid, or binding partner and thereby overcome any steric hindrance that can be presented by the support. The determination of an appropriate length of linker that allows for an optimal cellular response or lack thereof, can be determined by screening the Mtb polypeptide or encoding nucleic acid with varying linkers in the assays detailed in the present disclosure.

A composite support comprising more than one type of Mtb polypeptide or encoding nucleic acid is also envisioned. A “composite support” can be a carrier, a resin, or any macromolecular structure used to attach or immobilize two or more different Mtb polypeptides or encoding nucleic acids. As above, the insertion of linkers, such as λ linkers, of an appropriate length between the Mtb polypeptide or encoding nucleic acid and the support is also contemplated so as to encourage greater flexibility in the molecule and thereby overcome any steric hindrance that can occur. The determination of an appropriate length of linker that allows for an optimal cellular response or lack thereof, can be determined by screening the Mtb polypeptide or encoding nucleic acid with varying linkers in the assays detailed in the present disclosure.

In other embodiments, the multimeric and composite supports discussed above can have attached multimerized Mtb polypeptides or encoding nucleic acids so as to create a “multimerized-multimeric support” and a “multimerized-composite support”, respectively. A multimerized ligand can, for example, be obtained by coupling two or more Mtb polypeptides or encoding nucleic acids in tandem using conventional techniques in molecular biology. The multimerized form of Mtb polypeptides or encoding nucleic acids can be advantageous for many applications because of the ability to obtain an agent with a higher affinity, for example. The incorporation of linkers or spacers, such as flexible λ linkers, between the individual domains that make-up the multimerized agent can also be advantageous for some embodiments. The insertion of λ linkers of an appropriate length between protein binding domains, for example, can encourage greater flexibility in the molecule and can overcome steric hindrance. Similarly, the insertion of linkers between the multimerized Mtb polypeptides or encoding nucleic acids and the support can encourage greater flexibility and limit steric hindrance presented by the support. The determination of an appropriate length of linker can be determined by screening the Mtb polypeptides or encoding nucleic acid in the assays detailed in this disclosure.

Embodiments also include methods of using vaccine compositions and immunogen preparations comprising the Mtb polypeptides or encoding nucleic acids, or a truncated or mutated version thereof, and, optionally, an adjuvant. The next section describes some of these compositions in greater detail.

### Methods of Using the Vaccine Compositions and Immunogen Preparations

Routes of administration of the embodiments described herein include, but are not limited to, transdermal, parenteral, gastrointestinal, transbronchial, and transalveolar. Transdermal administration can be accomplished by application of a cream, rinse, gel, etc. capable of allowing the compositions described herein to penetrate the skin. Parenteral routes of administration include, but are not limited to, electrical or direct injection such as direct injection into a central venous line, intravenous, intramuscular, intraperitoneal, intradermal, or subcutaneous injection. Gastrointestinal routes of administration include, but are not limited to, ingestion and rectal. Transbronchial and transalveolar routes of administration include, but are not limited to, inhalation, either via the mouth or intranasally.

Compositions that are suitable for transdermal administration include, but are not limited to, pharmaceutically acceptable suspensions, oils, creams, and ointments applied directly to the skin or incorporated into a protective carrier such as a transdermal device (“transdermal patch”). Examples of suitable creams, ointments, etc. can be found, for instance, in the Physician's Desk Reference. Examples of suitable transdermal devices are described, for instance, in U.S. Pat. No. 4,818,540 issued Apr. 4, 1989 to Chinen, et al.

Compositions that are suitable for parenteral administration include, but are not limited to, pharmaceutically acceptable sterile isotonic solutions. Such solutions include, but are not limited to, saline, phosphate buffered saline and oil preparations for injection into a central venous line, intravenous, intramuscular, intraperitoneal, intradermal, or subcutaneous injection.

Compositions that are suitable for transbronchial and transalveolar administration include, but not limited to, various types of aerosols for inhalation. Devices suitable for transbronchial and transalveolar administration of these are also embodiments. Such devices include, but are not limited to, atomizers and vaporizers. Many forms of currently available atomizers and vaporizers can be readily adapted to deliver vaccines having ribavirin and an antigen.

Compositions that are suitable for gastrointestinal administration include, but not limited to, pharmaceutically acceptable powders, pills or liquids for ingestion and suppositories for rectal administration.

The nucleic acid constructs described herein, in particular, may be administered by means including, but not limited to, traditional syringes, needleless injection devices, or “microprojectile bombardment gene guns”. Alternatively, the genetic vaccine may be introduced by various means into cells that are removed from the individual. Such means include, for example, ex vivo transfection, electroporation, microinjection and microprojectile bombardment. After the gene construct is taken up by the cells, they are reimplanted into the individual. It is contemplated that otherwise non-immunogenic cells that have gene constructs incorporated therein can be implanted into the individual even if the vaccinated cells were originally taken from another individual.

According to some embodiments, the gene construct is administered to an individual using a needleless injection device. According to some embodiments, the gene construct is simultaneously administered to an individual intradermally, subcutaneously and intramuscularly using a needleless injection device. Needleless injection devices are well known and widely available. One having ordinary skill in the art can, following the teachings herein, use needleless injection devices to deliver genetic material to cells of an individual. Needleless injection devices are well suited to deliver genetic material to all tissue. They are particularly useful to deliver genetic material to skin and muscle cells. In some embodiments, a needleless injection device may be used to propel a liquid that contains DNA molecules toward the surface of the individual's skin. The liquid is propelled at a sufficient velocity such that upon impact with the skin the liquid penetrates the surface of the skin, permeates the skin and muscle tissue therebeneath. Thus, the genetic material is simultaneously administered intradermally, subcutaneously and intramuscularly. In some embodiments, a needleless injection device may be used to deliver genetic material to tissue of other organs in order to introduce a nucleic acid molecule to cells of that organ.

Preferred embodiments include methods of enhancing an immune response to a desired antigen by providing an animal in need with an amount of adjuvant (e.g., ribavirin) and one or more of the nucleic acid or polypeptide compositions disclosed herein that is effective to enhance said immune response. In these embodiments, an animal in need of an enhanced immune response to an antigen/target is identified by using currently available diagnostic testing or clinical evaluation. By one approach, for example, an individual at risk of becoming infected with Mtb, is provided with the vaccine compositions described above in an amount sufficient to elicit a cellular and humoral immune response to Mtb so as to protect said individual from becoming infected with Mycobacterium tuberculosis.

### Humoral Immune Response

Some embodiments relate to the use of the Mtb polypeptide polypeptides disclosed herein, according the methods above (e.g. using TAP or adapter technology) can be used to identify antigenic targets of humoral immunity in Mtb non-human animals and human patients. A humoral immune response relates to the generation of antibodies and their ability to bind to a particular antigen. In general, the humoral immune system uses white blood cells (B-cells), which have the ability to recognize antigens, to generate antibodies that are capable of binding to the antigens.

In one embodiment, the Mtb polypeptides of the invention are generated according to the methods described above. In certain aspects of this embodiment additional polynucleotide sequences that encode linker molecules are added to the TAP primary fragment or the TAP expression fragment such that the expressed Mtb polypeptides are fused to a linker molecule. As discussed previously, the term “linker molecule” encompasses molecules that are capable of immobilizing the polypeptides to a solid support.

In a particular embodiment, a Mtb polynucleotide of interest is fused to a HA epitope tag such that the expressed product can include the Mtb gene product fused to the HA epitope. In another embodiment, a Mtb polynucleotide of interest is combined with a histidine (His) coding sequence, such that the expressed product can include the Mtb gene product and a 6.times., 7.times., 8.times., 9.times., or 10.times.histidine tag. In other embodiments a Mtb polynucleotide is combined with a sequence that codes for a GST tag, fluorescent protein tag, or Flag tag. Using these methods it is possible to express and tag every Mtb polypeptide encoded by its genome. In another embodiment, the tagged Mtb polypeptide can be attached to a solid support, such as a 96-well plate. The immobilize polypeptides can be contacted with an antiserum or other fluid containing antibodies from an animal that has been immunized with one or more antigens from Mtb. In one embodiment, ELISA and Western blot assays are performed in parallel to detect the presence of immunogenic Mtb polypeptides.

As an example of an ELISA assay, tagged Mtb polypeptides can be immobilized on a solid support, such as a 96-well plate. The immobilized Mtb polypeptides are then incubated with serum from an animal that has been immunized with one or more antigens from Mtb, or has been infected directly with Mtb by inoculation, aerosol delivery, or the like. The reaction mixture can be washed to remove any unbound serum antibodies. The ability of the serum antibodies to bind to the bound Mtb polypeptides can then be detected using any one of a number of methods. For example, enzyme linked secondary antibodies can be added to detect the presence of an antigen specific antibody. Any enzyme linked secondary antibody can be used in this invention, depending on the source of the serum. For example, if vaccinated mouse serum is used to provide the primary antibody, enzyme linked anti-mouse antibody can be used as a secondary antibody Likewise if human serum is used to provide the primary antibody, enzyme linked anti-human serum can be used as a secondary enzyme.

Any suitable assay can be used to determine the amount of bound polypeptide specific antibody. Also, skilled artisans can develop the enzyme assay to determine the amount of polypeptide specific antibody that is bound. In one embodiment, the readout from an assay can show the presence of different levels of antibody in each of the 96 wells. For example, while some Mtb polypeptides are not able to elicit any serum antibodies, other Mtb polypeptides can elicit intermediate levels of antibodies, and some can elicit high antibody levels. In one embodiment, polypeptides that generate high antibody titers can be further researched to determine which polypeptides are present on the surface of the virus. In a particular embodiment of the invention Mtb polypeptides that generate high antibody titers and that are located on the surface of the virus are candidates for use in the development of a subunit Mtb vaccine.

In addition, serodiagnostic tests may be developed using antigens identified and characterized by these methods. That is, the peptide (epitopes) identified herein find use in detecting antibodies in serum from Mtb infected or exposed organisms, animals or patients.

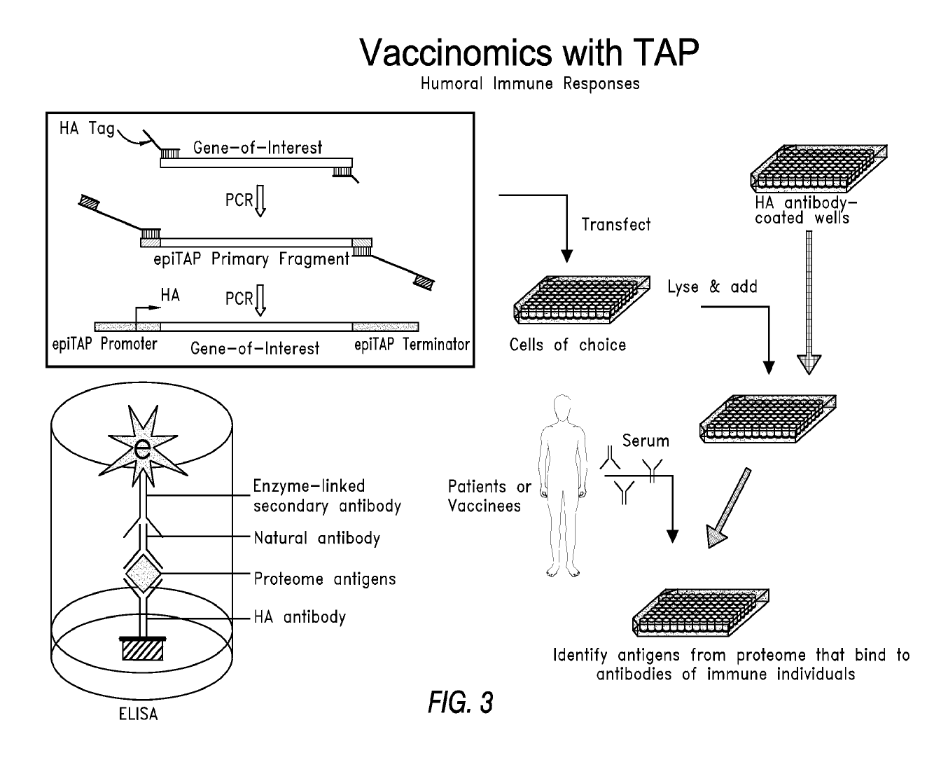

FIG. 3 demonstrates one embodiment of determining the humoral immune response generated by an array of polypeptides. One of skill in the art may deviate in certain details from those shown in FIG. 3. For example, the HA tag, or any other tag as described above, may be placed at either the C-terminal or N-terminal end of the polypeptide to insure that epitopes are not concealed due to binding to the plate. Instead of HA tagged polypeptides, a histidine tag can be used, and the polypeptides can be bound to nickel coated plates. For example a 6× 7×, 8×, 9×., or 10× histidine tag can be used. Alternatively, histidine tagged polypeptides can be purified from either transfected cells or from the in vitro transcription translation system. Furthermore, purified Mtb polypeptides can be attached non-specifically to polypeptide-absorbing plates such as Immulon plates, for example.

In one aspect, highly immunogenic Mtb antigens can be detected by comparing the results of Western blotting analysis with ELISA. Western blotting and ELISA are two independent yet complementary methods that may be used to detect immunogenic Mtb in qualitative and quantitative ways. Western blotting is often used to examine the quality of a polypeptide or protein sample, including such parameters as purity, protein integrity, and degradation. Western blotting detects polypeptides in their denatured form. In one aspect of this embodiment, ELISA, which detects native polypeptides, is used to further examine Western-positive Mtb polypeptides in a more quantitative fashion, to illustrate the strength of the Mtb epitope's immunogenicity.

### Cell-Mediated Immune Response

Mtb peptides disclosed herein can also be exploited to identify the highly immunogenic targets of cell-mediated immunity in Mtb vaccinated non-human animals. In contrast to a humoral immune response, where an antibody binds directly binding to an antigen, a cell-mediated immune response relates to T-cells binding to the surface of other cells that display the antigen. When certain T-cells come into contact with a presented antigen, they produce and release cytokines such as interferon-.gamma. (IFN-γ or Tumor Necrosis Factor-alpha (TNF-.α). Cytokines are cellular signals that can alter the behavior or properties of another cell. For example, cytokines may inhibit viral replication, induce increased expression of MHC class I and peptide transporter molecules in infected cells, or activate macrophages. Accordingly, cytokines released by T-cells, associated with the binding to an antigen, can be used to identify and detect T-cell/antigen interactions.

Some cells have MHC molecules on their membranes to present antigens to T-cells. Efficient T-cell function relies on proper recognition of the MHC-antigen complex. There are two types of MHC molecules: Class I and Class II. The two different classes of MHC molecules bind peptides from different sources inside the cell for presentation at the cell surface to different classes of T-cells. Any T-cell can be used in the embodiments disclosed herien, and include for example both CD4⁺ and CD8⁺ T-cells. CD8⁺ cells (cytotoxic T-cells) bind epitopes that are part of class I MHC molecules. CD4⁺ T-Cells, which includes inflammatory CD4 T-cells and helper CD4 T-cells, bind epitopes that are part of class II MHC molecules. Only specialized antigen-presenting cells express class II molecules.

There are three main types of antigen-presenting cells: B cells, macrophages and dendritic cells. Each of these cell types is specialized to process and present antigens from different sources to T-cells, and two of them, the macrophages and the B cells, are also the targets of subsequent actions of armed effector T-cells. These three cell types can express the specialized co-stimulatory molecules that enable them to activate naive T-cells, although macrophages and B cells express those molecules only when suitably activated by infection.

Embodiments relate to detecting Mtb polypeptides capable of evoking a cell-mediated immune response in order to identify potential candidates for use in a subunit vaccine or other pharmaceutical composition. According to one method of detecting a cell-mediated immune response, an Mtb polypeptide is delivered to an antigen-presenting cell where it can be presented in a manner that is recognized by antigen specific T-cells. In another embodiment, a transcriptionally active gene as disclosed herein can be delivered to an antigen-presenting cell where expressed and presented in a manner that can be recognized by antigen specific T-cells. Mtb antigen specific T-cells can be acquired from numerous sources. For example, animals that have been infected, or immunized with one or more antigens from Mtb virus are a good source of antigen specific T-cells. Alternatively, human Mtb patients and volunteers immunized with Mtb can be a source of antigen specific T-cells.

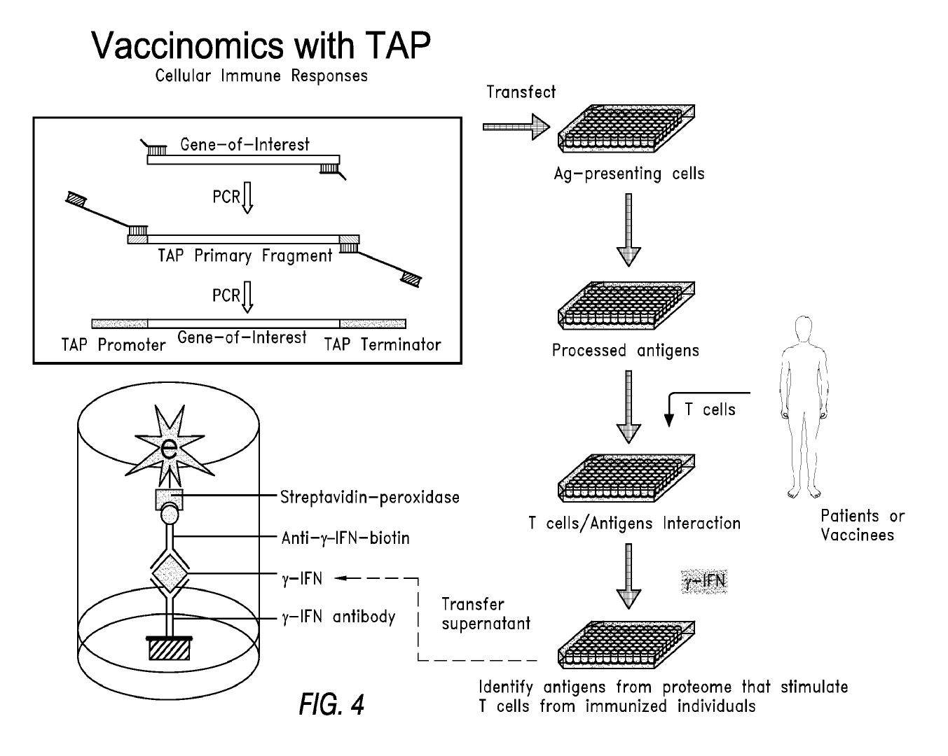

FIG. 4 demonstrates one embodiment of determining the cell-mediated immune response generated by an array of polypeptides. One of skill in the art may deviate in certain details from those shown in FIG. 4.

In order to test the ability of Mtb polypeptides to elicit a cell-mediated response, a plurality of Mtb polynucleotides can be amplified and made transcriptionally active using TAP technology. In one embodiment about 10, about 20, about 30, about 40, about 50, about 60, about 70, about 80, about 90, about 100, about 110, about 120, about 130, about 140, about 150, about 160, about 170, about 180, about 190, about 200, about 210, about 220, about 230, about 240, about 250, about 260, about 266 Mtb polynucleotides are made transcriptionally active using TAP technology, as described herein.

In one embodiment, transcriptionally active Mtb polynucleotides can be transfected into an antigen-presenting cell and expressed within the cell. In another embodiment, instead of transfecting the genes into an antigen-presenting cell, the Mtb TAP fragments can be expressed in an in vivo or in vitro (cell-free) expression system and the expressed polypeptide can be delivered into the antigen-presenting cell. The polypeptide can be delivered into the antigen-presenting cell according to any method. In one embodiment, the polypeptide can be delivered using the technology described in U.S. patent application Ser. No. 09/738,046, entitled “Intracellular Protein Delivery Reagent” and U.S. patent application Ser. No. 10/141,535, entitled “Intracellular Protein Delivery Compositions and Methods of Use,” both of which are hereby incorporated by reference in their entirety. The reagents described therein are capable of delivering any type of polypeptide into any type of cell.

In certain embodiments, reagents used to deliver polypeptides into cultured cells can be a cationic lipid formulation. In one embodiment, these reagents can deliver fluorescently labeled antibodies, high and low molecular weight dextrans, phycoerythrin-BSA, caspase 3, caspase 8, granzyme B, and .beta.-galactosidase into the cytoplasm of a variety of different adherent and suspension cells. Caspases delivered to cells with are functional, since they can be shown to send cells into apoptosis. In one embodiment, Mtb polypeptides are delivered into dendritic cells using these reagents.

Detecting a T-cell's ability to bind to an antigen-presenting cell, after the antigen-presenting cell has processed a particular polypeptide, is useful in determining whether the particular polypeptide evokes a cell-mediated immune response. Once a particular polypeptide is delivered into or expressed in the antigen-presenting cell, an assay can be performed to identify T-cell interaction with the MHC-antigen complex. In one embodiment, it can be determined if T-cells obtained from an animal that was immunized with Mtb can bind to a particular antigen presented by an antigen-presenting cell. For example, an ELIspot assay (Enzyme-Linked Immuno spotting; ELIspot) can be performed to identify antigen specific T-cells. Similar immunoassays can be performed to identify Mtb antigens (presented by an antigen-presenting cells) that stimulate T-cells from active Mtb patients or immunized individuals.

One method of detecting a T-cell/antigen interaction is to measure the amount of a particular cytokine released by the T-cell when it interacts with a MHC-antigen complex. The skilled artisan can appreciate that other cellular signals can be used to indicate a cell-mediated immune response. In one embodiment, the levels of IFN-.gamma. released by T-cells can indicate whether a particular peptide is capable of evoking a cell-mediated immune response. In a particular embodiment, an antibody specific for IFN-.gamma. can be coated onto a solid support. Unbound antibodies can be washed away and IFN-.gamma. obtained from the supernatant containing T-cells plus antigen-presenting cells or antigen transduced antigen-presenting cells, can be added to the wells. A biotinylated secondary antibody specific for IFN-γ can be added. Excess secondary antibody can be removed and Streptavidin-Peroxidase can be added to the mixture. Streptavidin-Peroxidase is capable of binding to the biotinylated antibody to complete the four-member immunoassay “sandwich.” Excess or unbound Streptavidin-Peroxidase is easily removed from the mixture. In order to detect amount of bound Streptavidin-Peroxidase, a substrate solution can be added which reacts with the Streptavidin-Peroxidase to produce color. The intensity of the colored product is directly proportional to the concentration of IFN-.gamma. present in the T-cell/antigen-presenting cell supernatant. Kits for performing these types of immunoassay are readily available from many commercial suppliers or the necessary reagents composing such kits can be purchased separately or produced in-house. In one embodiment, processed and presented Mtb polypeptide that evokes T-cells to produce a high level of IFN-.gamma. can be considered a strong candidate for use in developing a subunit vaccine.

Those with skill in the art will appreciate that other methods can be used to detect T-cell/Antigen interactions. These methods include bead based assays, flow-based assays, RT-PCR based assays, cytokine ELISAs, lymphoproliferation assays, cytotoxic T cell assays, or any other assay that can detect the interaction of a T-cell with a responder cell (e.g. macrophage).

### EXAMPLES

The following examples are provided to demonstrate particular situations and settings in which this technology may be applied and are not intended to restrict the scope of the invention and the claims included in this disclosure.

### Example 1

### General Procedure for Generating Histidine Tagged TAP Express Fragments

This example describes the general methodology used to generate a library of M. tuberculosis peptides, which can be used in screening assays described below.

Briefly, the following procedure was used to produce tagged T7 TAP fragments: Mtb genes are amplified from genomic DNA (Mtb strain H37Rv). A first PCR reaction is run with customized 5′ and 3′ primers. The 5′ primers contain between 43-48 bases. Preferably, the T-7-His TAP ends contain 28 bases while the gene-specific component contained between 15-20 bases. The 3′ primers contain between 45-50 bases. Preferably, the T7-terminator TAP ends contained 30 bases while the gene specific component contains between 15-20 bases. The reaction temperature and times for the first PCR reaction are: 94° C. for 2 minutes, followed by 28 cycles of: 94° C. for 20 seconds, 58° C. for 35 seconds, and 70° C. for 2 minutes (for genes that contained more than 2 kb, 1 minute is added for each kb).

After the first PCR reaction is performed, an aliquot of each PCR reaction from the previous step is transferred into a PCR reaction containing the T7-histidine promoter fragment and T7 terminator fragment. Preferably, the T7 promoter primer contains 25 bases, while the T7-promoter-His tag fragment contains a 104 base EcoRV/BglII fragment. The T7-terminator fragment is a 74 base oligonucleotide. The reaction temperature and times for the second PCR reaction are: 94° C. for 2 minutes, followed by 30 cycles of: 94° C. for 20 seconds, 60° C. for 35 seconds, and 70° C. for 2 minutes (for genes that contained more than 2 kb, 1 minute was added for each kb).

This reaction was used to generate candidate M. tuberculosis peptides.

### Example 2

### Using the Mtb Proteome to Identify the Antigenic Targets of Humoral Immunity in Humans

The following method was used to systematically screen and identify antigens in Mtb that give rise to a protective humoral immune response in humans. A bioinformatics approach was used to order the M. tuberculosis polynucleotide sequences for amplification. The Mtb genome was first analyzed for hydrophobicity by the method of Doolittle. Hydrophilic polynucleotides sequences were then further grouped by size. Hydrophilic open reading frames/coding regions longer than 500 bp were selected for TAP amplification. Initially, three hundred Mtb genes were synthesized by TAP and ˜3200 proteins were translated and purified in arrays, as described below.

### TAP PCR

The PCR reactions were performed such that a nucleotide sequence encoding a 6×His tag was fused to these amplified transcriptionally active genes. The resulting His tagged TAP fragments were expressed to produce ˜3200 Mtb polypeptides containing the His tag.

A detailed procedure that was used to produce tagged T7-TAP Express fragments is as follows: groups of Mtb polynucleotide sequences were amplified from Mtb genomic DNA. A first PCR reaction was performed using customized 5′ and 3′ primers. The 5′ primers contained between 43-48 bases. In particular, the T-7-His TAP ends contained 28 bases while the gene-specific component contained between 15-20 bases. The 3′ primers contained between 45-50 bases. Specifically, the T7-terminator TAP ends contained 30 bases while the gene specific component contained between 15-20 bases.

The PCR reactions contained 100 ng Mtb genomic DNA, 25 nM final concentration of 5′ and 3′ primers. Polymerase, PCR buffer and nucleotides were from Clontech. The reaction temperature and times for the first PCR reaction were: 94° C. for 2 minutes, followed by 30 cycles of: 94° C. for 30 seconds, 48° C. for 1 min., and 68° C. for 2.5 minutes.

Following the first PCR reaction, an aliquot of each PCR reaction containing 100 ng of PCR product from the previous step was transferred into a PCR reaction containing the TAP promoter and terminator fragments. The sequences of these fragments were:

Promoter fragment:
(SEQ ID NO: 27)
5′CGGTCACGCTTGGGACTGCCATAGGCTGGCCCGGTGATGCCG
GCCACGATGCGTCCGGCGTAGAGGATCGAGATCTCGATCCCGCGAAATTA
ATACGACTCACTATAGGGAGACCACAACGGTTTCCCTCTAGAAATAATTT
TGTTTAACTTTAAGAAGGAGATATACC 3′
Terminator fragment:
(SEQ ID NO: 28)
5′GGGGGGGGTTCTCATCATCATCATCATCATTAATAAAAGGGC
GAATTCCAGCACACTGGCGGCCGTTACTAGTGGATCCGGCTGCTAACAAA
GCCCGAAAGGAAGCTGAGTTGGCTGCTGCCACCGCTGAGCAATAACTAGC
ATAACCCCTTGGGGCCTCTAAACGGGTCTTGAGGGGTTTTTTGCTGAAAG
GAGGAACTATATCCGGAGCGACTCCCACGGCACGTTGGCAAGCTCG 3′

The reaction temperature and times for the second PCR reaction were: 94° C. for 2 minutes, followed by 30 cycles of: 94° C. for 30 seconds, 48° C. for 60 seconds, and 68° C. for 2.5 minutes.

### Protein Expression

The TAP fragments generated by PCR were used as templates for in vitro protein expression using a Roche RTS100 transcription/translation kit according to manufacturer's instructions. Approximately 0.5˜1.0 μg PCR product was used as template, producing approximately 0.5˜5.0 μg of protein per template.

### Protein Purification

MAGNEHIS™ nickel-coated magnetic beads (Promega) were used to purify the expressed proteins. 15 μl of Ni-magnetic beads (Promega) were pipetted into each well of a microtiter plate. To each well 50 μl wash buffer (50 mM NaHPO₄, pH8.0, 300 mM NaCl, 100 mM imidazole) was added with mixing and the plates were placed on a magnetic stand. The supernatant was removed and wash was repeated. 50 μl of the Protein mixture was added with gentle pipetting. The mixture was incubated at room temperature for 2 minutes. The beads were then separated using a magnetic stand, washed 3 times with 150 μl wash buffer and the bound protein was eluted from the beads with 50 μl of 50 mM NaHPO₄, pH 8.0, 300 mM NaCl, 250 mM imidazole.

### Western Blot.

15 μl of the purified proteins were resolved on 4-12% SDS-polyacrylamide gels and transferred to nitrocellulose membranes The membranes were blocked in TBST/1% BSA, followed by incubation with TBST/1% BSA containing 1000-fold diluted IgG isolated from pooled sera from TB patients. The blots were washed and then incubated with alkaline phosphatase-conjugated goat-anti human serum secondary antibody. Colorimetric development was used to develop the blots. Of the ˜3200 Mtb polypeptides evaluated, 72 were positive in a Western blot using pooled sera from TB patients.

In order to eliminate Mtb polypeptides from the screen that are present in healthy individuals, Mtb polypeptides that tested positive with pooled IgG isolated from sera from TB patients were tested using a similar Western blot procedure as described above, except that instead of using IgG from pooled sera from TB patients, IgG isolated from pooled sera from healthy individuals was used as the primary antibody. Of the 72 polypeptides that tested positive with sera from TB patients, 53 were also recognized by IgG antibodies from the pooled sera from healthy patients.

In order to eliminate Mtb polypeptides from the screen that are present in non-tuberculosis mycobacteria, the remaining 19 Mtb polypeptides, recognized by IgG isolated from sera of TB, but not healthy patients, were subjected to a third screen. The remaining 19 polypeptides were tested in a Western blot as described above, except that instead of using IgG from pooled sera from healthy individuals, IgG isolated from pooled sera from individuals infected with non-tuberculosis mycobacteria (“NTM”). Of the 19 Mtb polypeptides tested, 7 were recognized by the IgG isolated from pooled sera from individuals infected with NTM. The remaining 12 Mtb polypeptides are thus useful as antigens/biomarkers.

The 12 Mtb polypeptides identified in this example are provided as SEQ ID NO's: 1-12. Their encoding nucleic acids are provided herein as SEQ ID NO's: 13-24. This example demonstrates that the Mtb polypeptides and Mtb polynucleotides disclosed herein are useful in immunogenic compositions for prophylactic purposes, i.e., to generate protective immune responses against Mtb, as well as in diagnostic settings.

### Example 3

### Using the Mtb Proteome to Identify the Antigenic Targets of Cell-Mediated Immunity in Mtb Vaccinated Mice and Humans

The following is a method that is used to systematically screen and identify antigens in Mtb that give rise to a protective cell-mediated immune response. Through the use of TAP technology coding sequences of the Mtb genome are amplified. The PCR reactions are performed such that each amplified coding sequence becomes transcriptionally active. The resulting TAP fragments are expressed to produce Mtb polypeptides. Each of the polypeptides is delivered into dendritic cells, located in 96-well plates, using a polypeptide delivery reagent. Serum from Mtb immunized humans is added to each of the different wells.

An IFN-γ ELIspot assay is run using the following materials and method:

### Materials:

Millipore 96-well multi-screen filtration plates (Millipore #MAIP S45-10) (Millipore, Bedford, Mass.)

Anti-IFN-□ purified MAb (Clone 1-D1K) (MABTECH #3420-3) (Mabtech, Naka, Sweden)

Anti-IFN-g Biotinylated MAb (Clone 7-B6-1) (MABTECH #3420-6) (Mabtech, Naka, Sweden)

Streptavidin-Alkaline Phosphatase (MABTECH #3310-8) (Mabtech, Naka, Sweden)

Alkaline Phosphate Substrate Kit (BIO-RAD #170-6432) (Bio-Rad, Hercules, Calif.)

Carbonate Buffer pH 9.6 (0.2 μM sterile filtered)

RPMI-1640 Medium (GIBCO #22400-089) (Gibco, Grand Island, N.Y.)

Fetal Bovine Serum (Sigma #F4135-500 mL) (Sigma, St. Louis, Mo.)

1×PBS (Prepared from 10×PBS DIGENE #3400-1010) (DIGENE, Gaithersburg, Md.)

Tween® 20 (J. T. Baker #X251-07) (J. T. Baker, Phillipsburg, N.J.)

### Method:

96-well plates are coated with Coating Antibody (anti-IFN-g Clone 1-D1K) at 10-15 μg/mL (100 μL/well) and incubated at 4° C. overnight. Using aseptic technique, plates are flicked to remove Coating Antibody and washed 6 times with RPMI-1640. Plates are blocked with 100 μL/well of RPMI-1640+10% FBS (or Human AB serum) for 1-2 hours at room temperature. Plates are flicked to remove blocking buffer and 100 μL/well of antigen specific or control peptides are added at a final concentration of 10 μg/well. Peripheral blood lymphocytes (PBL) are added at 4×105/well and 1×105/well. Plates are incubated at 37° C./5% CO₂ for 36 hours. Plates are flicked to remove cells and washed 6 times with PBS+0.05% Tween® 20 at 200-250 μL/well. Plates are blot dried on paper towels.

Biotinylated antibody (anti-IFN-g Clone 7-B6-1) diluted 1:1,000 in 1×PBS at 100 μL/well is added. The resulting solution is incubated for 3 hours at room temperature. Plates are flicked to remove biotinylated antibody and washed 6 times with PBS+0.05% Tween® 20 at 200-250 μL/well. Plates are blot dried on paper towels. Streptavidin alkaline phosphatase is added at 100 μL/well diluted 1:1,000 in 1×PBS. The plates are incubated for 1 hour at room temperature. Plates are flicked to remove the streptavidin alkaline phosphatase and washed 6 times with 0.05% Tween® 20 at 200-250 μL/well. The plates are washed again 3 times with 1×PBS at 200-250 μL/well. The plates are blot dried on paper towels.

Substrate is added at 100 μL/well for 10-15 minutes at room temperature. The substrate is prepared according to manufacturer's protocol. The 25× substrate buffer is diluted in dH20 to a 1× concentration. Reagent A & B are each diluted 1:100 in the 1× substrate buffer. Rinsing plates with generous amounts of tap water (flooding plate and flicking several times) stops colorimetric substrate. Plates are allowed to dry overnight at room temperature in the dark. Spots corresponding to IFN-γ producing cells are determined visually using a stereomicroscope (Zeiss KS ELIspot). Results can be expressed as the number of IFN-γ-secreting cells per 10⁶ spleen cells. Responses are considered positive if the response to test Mtb peptide epitope is significantly different (p<0.05) as compared with the response to no peptide and if the stimulation index (SI=response with test peptide/response with control peptide) is greater than 2.0.

### Example 4

### Peptide Vaccine Against M. tuberculosis

An individual at risk of becoming infected with M. tuberculosis is identified using routine clinical methods. The individual is administered an immunogenic composition that includes one or more Mtb polypeptides of SEQ ID NO's: 1-12, or an immunogenic fragment thereof in combination with an adjuvant.

### Example 5

### DNA Vaccine Against M. tuberculosis

An individual at risk of becoming infected with M. tuberculosis is identified using routine clinical methods. The individual is administered an immunogenic composition that includes one or more Mtb polynucleotides as described herein, in an expression system to of SEQ ID NO's: 13-24, or an immunogenic fragment thereof in combination with an adjuvant.

What is claimed is:

1. An isolated polynucleotide encoding an antigenic Mtb polypeptide, wherein the polynucleotide is selected from the group consisting of:
(a) a polynucleotide sequence comprising of any one of SEQ ID NOs: 13-24;
(b) a fragment of the polynucleotide sequence of (a), wherein the fragment encodes an antigenic peptide epitope; and
(c) a polynucleotide that is at least 90% identical to the full length polynucleotide of (a) or (b), wherein said polynucleotide encodes an antigenic peptide epitope.

2. The isolated polynucleotide of claim 1, wherein said mammal is a rabbit, human, guinea pig, or mouse.

3. The isolated polynucleotide of claim 1, wherein the antigenic polypeptide is immunogenic.

4. The Mtb polynucleotide of claim 1, further comprising a promoter operably coupled to the Mtb polynucleotide.

5. The Mtb polynucleotide of claim 1, further comprising a terminator operably coupled to the Mtb polynucleotide.

6. An isolated Mtb polypeptide selected from the group consisting of:
(a) a polypeptide comprising the amino acid sequence of any one of SEQ ID NOs: 1-12;
(b) a fragment of the polypeptide of (a), wherein said fragment comprises an antigenic epitope; and
(c) a polypeptide that is at least 90% identical to the full length polypeptide of (a) or the full length fragment of (b), wherein said polypeptide or fragment comprises an antigenic epitope.

7. The isolated Mtb polypeptide of claim 6, further comprising an affinity tag operably coupled to the amino terminus or the carboxy terminus.

8. The isolated Mtb polypeptide of claim 7, wherein the affinity tag is selected from the group consisting of an HA tag or a His tag.

9. A composition comprising at lest one isolated Mtb polypeptide selected from the group consisting of SEQ ID NOs: 1-12, or an immunogenic fragment thereof, and a pharmaceutically acceptable carrier.
